(19) United States
(12) Patent Application Publication
(10) Pub. No.: US 2004/0266743 A1
(43) Pub. Date: Dec. 30, 2004

(54) Combination of an aldosterone receptor antagonist and a renin inhibitor

(75) Inventors: Ellen G. McMahon, St. Louis, MO (US); Amy E. Rudolph, Fairfield, CT (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/834,708

(22) Filed: Apr. 29, 2004

Related U.S. Application Data
(60) Provisional application No. 60/469,583, filed on May 9, 2003.

(51) Int. Cl. A61K 31/585; A61K 31/56

(52) U.S. Cl. 514/171

(57) ABSTRACT

Novel combinations, compositions, and therapeutic methods of treatment of a hypertension, cardiovascular disease, renal dysfunction, edema, cerebrovascular disease, or insulinopathy pathological condition in a subject, wherein the methods comprise the administration of a combination of one or more aldosterone receptor antagonists and one or more renin inhibitors.

### CROSS-REFERENCE TO RELATED APPLICATION

This application claims priority from U.S. application Ser. No. 60/469,583, filed May 9, 2003.

### FIELD OF THE INVENTION

Combinations of an aldosterone receptor antagonist and a renin inhibitor, compositions thereof, and therapeutic methods are described for use in the treatment or prevention of a pathological condition.

### BACKGROUND OF THE INVENTION

The renin-angiotensin-aldosterone system (“RAAS”) is one of the hormonal mechanisms involved in regulating pressure/volume homeostasis and also in the development of hypertension, a condition which can progress to more serious cardiovascular diseases such as congestive heart failure. Activation of the renin-angiotensin-aldosterone system begins with secretion of the enzyme renin from the juxtaglomerular cells in the kidney.

Renin is a natural enzyme that passes from the kidneys into the blood where it cleaves angiotensinogen to generate the decapeptide angiotensin I. Angiotensin I is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Angiotensin II also produces other physiological effects such as stimulating aldosterone secretion, promoting sodium and fluid retention, inhibiting renin secretion, increasing sympathetic nervous system activity, stimulating vasopressin secretion, causing a positive cardiac inotropic effect and modulating other hormonal systems. Inhibitors of the enzymatic activity of renin bring about a reduction in the formation of angiotensin I. As a result a smaller amount of angiotensin II is produced. Thus, renin inhibitors effectively reduce angiotensin II production and blood pressure.

A common problem associated with many renin inhibitors is that they are poorly absorbed from the gastrointestinal tract and, therefore, not suitable for treatment of chronic illnesses. Recently, however, aliskiren (Speedel, Basel, Switzerland), a renin inhibitor, was shown to have good bioavailability when administered orally, and is in clinical trials for the treatment of hypertension, congestive heart failure and chronic renal failure. A study in healthy volunteers showed that aliskiren administered once daily could inhibit all factors of the RAAS cascade in a dose-dependent manner. Maximum inhibition of plasma renin activity was achieved within one hour of administration and angiotensin I and angiotensin II were also inhibited in a dose-dependent manner. Furthermore, decreases in plasma and urinary aldosterone levels were detected after administration and natriuresis was also enhanced. These clinical data on aliskiren indicate that it can be an effective blood pressure-lowering agent, as well as a possible treatment for congestive heart failure and chronic renal failure. (Current Opinion in Investigational Drugs, 2002, Vol. 3 No. 10, pp. 1479-1482).

Goschke et al., U.S. Pat. No. 5,559,111, disclose compounds, including aliskerin, having renin-inhibiting properties that can be used as antihypertensive medicinal active ingredients.

Aldosterone is the body's most potent known mineralocorticoid hormone. As connoted by the term mineralocorticoid, this steroid hormone has mineral-regulating activity. It promotes sodium (Na⁺) reabsorption in epithelial cells through binding and activating the mineralocorticoid receptor (MR). Aldosterone increases sodium and water reabsorption in the distal nephron and promotes potassium (K⁺) and magnesium (Mg²⁺) excretion.

Aldosterone also can produce responses in nonepithelial cells. In fact, aldosterone receptors have been recently identified in brain tissue, heart tissue and blood vessels. Aldosterone-mediated responses in these tissues can have adverse consequences on the structure and function of the cardiovascular system. Hence, inappropriate aldosterone exposure can contribute to organ damage in disease settings.

The effect of aldosterone can be reduced through the use of an aldosterone receptor antagonist. Spironolactone, also known as ALDACTONE® (Pharmacia, Chicago, Ill.), is an example of an aldosterone receptor antagonist. According to United States Pharmacopeia, Rockville, Md., spironolactone is indicated for the management of essential hypertension, primary aldosteronism, hypokalemia, and edematous conditions such as congestive heart failure, cirrhosis of the liver, and nephrotic syndrome. The administration of spironolactone to severe heart failure patients was evaluated in the Randomized Aldactone Evaluation Study (RALES). RALES was a randomized, double-blinded, placebo-controlled trial that enrolled participants who had severe heart failure and a left ventricular ejection fraction of no more than 35% and who were receiving standard therapy, which typically included an angiotensin-converting enzyme inhibitor, a loop diuretic, and, in some cases, digoxin. The RALES subjects treated with spironolactone had a statistically significant reduction in mortality and incidence of hospitalization relative to placebo-treated subjects. New England Journal of Medicine 341, 709-717 (1999).

A class of steroidal-type aldosterone receptor antagonists exemplified by epoxy-containing spirolactone derivatives is described in U.S. Pat. No. 4,559,332 issued to Grob et al. This patent describes 9α, 11α-epoxy-containing spirolactone derivatives as aldosterone receptor antagonists that are useful for the treatment of hypertension, cardiac insufficiency and cirrhosis of the liver. One of the epoxy-steroidal aldosterone receptor antagonist compounds described in U.S. Patent 4,559,332 is eplerenone (also known as epoxymexrenone). Eplerenone is an aldosterone receptor antagonist that has a higher specificity for the MR compared to spironolactone.

Another class of steroidal-type aldosterone receptor antagonists is exemplified by drospirenone. Developed by Schering A G, this compound is an antagonist of mineralocorticoid and androgenic receptors, while also possessing progestagenic characteristics.

Additional uses of aldosterone receptor antagonists have been disclosed in the literature. Williams et al., WO 01/95892 and WO 01/95893, describe methods for the treatment of aldosterone-mediated pathogenic effects in a subject using an aldosterone receptor antagonist (including spironolactone and/or eplerenone). Rocha et al., WO 02/09683, describe methods of using an aldosterone receptor antagonist (including eplerenone and/or spironolactone) for the treatment of inflammation in a subject.

Therapies comprising the administration of an aldosterone receptor antagonist in combination with several other pharmacologically active compounds have been reported in the literature.

Egan et. al., WO 96/40255, disclose a combination treatment therapy utilizing an epoxy-steroidal aldosterone receptor antagonist and an angiotensin II antagonist for treating cardiofibrosis.

Alexander et al., WO 96/40257, disclose a combination treatment therapy utilizing an epoxy-steroidal aldosterone receptor antagonist and an angiotensin II antagonist for treating congestive heart failure.

Perez et al., WO 00/27380, disclose a combination treatment therapy utilizing an angiotensin converting enzyme inhibitor and an aldosterone receptor antagonist for reducing morbidity and mortality resulting from cardiovascular disease.

Alexander et al., WO 00/51642, disclose a combination treatment therapy utilizing an angiotensin converting enzyme inhibitor and an epoxy-steroidal aldosterone receptor antagonist for treating cardiovascular disease.

Alexander et al., WO 02/09760, disclose a combination therapy utilizing an epoxy-steroidal aldosterone receptor antagonist and a beta-adrenergic antagonist for treating circulatory disorders, including cardiovascular disorders such as hypertension, congestive heart failure, cirrhosis and ascites.

Schuh, WO 02/09761, disclose a combination treatment therapy utilizing an epoxy-steroidal aldosterone receptor antagonist and a calcium channel blocker for treating hypertension, congestive heart failure, cirrhosis and ascites.

Rocha et al., WO 02/09759, disclose a combination treatment therapy utilizing an epoxy-steroidal aldosterone receptor antagonist and a cyclooxygenase-2 inhibitor for treating inflammation related cardiovascular disorders.

Garthwaite et al., WO 01/87284, disclose a combination treatment therapy utilizing an epoxy-steroidal aldosterone receptor antagonist and a second anti-hypertensive agent in a delayed-release formulation for administration to a subject exhibiting a diurnal cycle of plasma aldosterone concentration.

Improved drug therapies for the treatment of subjects suffering from or susceptible to a pathological condition are highly desirable. In particular, there still is a need for drug therapies that (1) provide better control over pathological conditions, (2) further reduce pathological risk factors, (3) provide improved treatment and/or prevention of pathological conditions, (4) are effective in a greater proportion of subjects suffering from or susceptible to a pathological condition, particularly in those subjects who do not satisfactorily respond to conventional drug therapies, and/or (5) provide an improved side-effect profile relative to conventional drug therapies.

### SUMMARY OF THE INVENTION

The present invention is directed to a method for the treatment of a pathological condition in a subject which comprises administering an aldosterone receptor antagonist and a renin inhibitor for the treatment of a pathological condition.

The invention is further directed to a combination comprising an aldosterone receptor antagonist and a renin inhibitor in a pharmaceutically acceptable carrier.

The present invention is further directed to a method for the treatment of a pathological condition in a subject which comprises administering an aldosterone receptor antagonist and a renin inhibitor for the treatment of a pathological condition. The aldosterone receptor antagonist further exhibits a release profile, determined using a suitable release profile test, in which more than about 20% by weight of the aldosterone receptor antagonist is released at about four hours after initiation of the test.

The invention is further directed to a pharmaceutical composition comprising a first amount of an aldosterone receptor antagonist, a second amount of a renin inhibitor, and a pharmaceutically acceptable carrier.

The invention is further directed to a pharmaceutical composition comprising a first amount of an aldosterone receptor antagonist, a second amount of a renin inhibitor, and a pharmaceutically acceptable carrier, wherein the aldosterone receptor antagonist exhibits a release profile, determined using a suitable release profile test, in which more than about 20% by weight of the aldosterone receptor antagonist is released at about four hours after initiation of the test.

The invention is further directed to a kit containing a first amount of an aldosterone receptor antagonist and a second amount of a renin inhibitor.

The invention is further directed to a kit containing a first amount of an aldosterone receptor antagonist and a second amount of a renin inhibitor. The aldosterone receptor antagonist exhibits a release profile, determined using a suitable release profile test, in which more than about 20% by weight of the aldosterone receptor antagonist is released at about four hours after initiation of the test.

Other aspects of the invention will be in part apparent and in part pointed out hereinafter.

### BRIEF DESCRIPTION OF THE DRAWINGS

FIG. 1 illustrates the interrelationship of the Renin-Angiotensin-Aldosterone System, Neutral Endopeptidase System, and Kallikrein-Kinin System.

### DETAILED DESCRIPTION OF THE PREFERRED EMBODIMENTS

The present invention relates to combinations, compositions, and methods to treat or prevent one or more pathological conditions in a subject through the therapeutical administration of an aldosterone receptor antagonist in combination with a renin inhibitor.

In one embodiment, the aldosterone receptor antagonist is an epoxy-steroidal aldosterone receptor antagonist. In another embodiment, the aldosterone receptor antagonist is an epoxy-steroidal aldosterone receptor antagonist containing a 9,11-epoxy moiety. In still another embodiment, the aldosterone receptor antagonist is pregn-4-ene-7, 21-dicarboxylic acid, 9,11-epoxy-17-hydroxy-3-oxo-,γ-lactone, methyl ester, (7α, 11α, 17α)—(also known as eplerenone or epoxymexrenone) or salt thereof, including, but not limited to mono- and di-salts thereof, hereinafter collectively referred to as “eplerenone.”

In another embodiment, the aldosterone receptor antagonist is a spirolactone-type aldosterone receptor antagonist, such as spironolactone.

In another embodiment, the aldosterone receptor antagonist is selected from the group consisting of eplerenone and spironolactone.

Indications

The pathological conditions that can be treated or prevented in accordance with the present invention include, but are not limited to, hypertension, cardiovascular disease, renal dysfunction, liver disease, cerebrovascular disease, vascular disease, retinopathy, vasculopathy, neuropathy (such as peripheral neuropathy), organ damage, insulinopathy, edema, endothelial dysfunction, baroreceptor dysfunction, migraine headaches, hot flashes, premenstrual tension, and the like. Cardiovascular disease includes, but is not limited to, heart failure, congestive heart failure, cardiac hypertrophy, acute heart failure, arrhythmia, diastolic dysfunction (such as left ventricular diastolic dysfunction, diastolic heart failure, and impaired diastolic filling), systolic dysfunction, ischemia, hypertrophic cardiomyopathy, sudden cardiac death, myocardial and vascular fibrosis, restinosis after angioplasty, myocardial dysfunction during or following a myocardial infarction, impaired arterial compliance, myocardial necrotic lesions, vascular damage, stroke, myocardial infarction, left ventricular hypertrophy, decreased ejection fraction, cardiac lesions, vascular wall hypertrophy, endothelial thickening, fibrinoid necrosis of coronary arteries, hyperaldosteronism, anxiety states, and the like. Renal dysfunction includes, but is not limited to, renal failure, glomerulosclerosis, end-stage renal disease, acute renal failure, renal impairment following treatment with cyclosporine or other immunosuppressants diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions of affecting glomeruli and microvessels), and the like. Liver disease includes, but is not limited to, liver cirrhosis, liver ascites, hepatic congestion, and the like. Cerebrovascular disease includes, but is not limited to stroke. Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like. Edema includes, but is not limited to, peripheral tissue edema, hepatic congestion, splenic congestion, liver ascites, respiratory or lung congestion, and the like. Insulinopathies include, but are not limited to, insulin resistance, Type I diabetes mellitus, Type II diabetes mellitus, glucose sensitivity, pre-diabetic state, syndrome X, and the like. Gastroenteric disorders such as diarrhea and hyperchlorhydria, irritable bowel syndrome. Endocrine and metabolic disease such as obesity hyperaldosteronemia, glaucoma, hypertensive or diabetic retinopathy, elevated intraocular pressure, and the like. Autoimmune disease such as rheumatism.

In one embodiment the pathological condition is selected from the group consisting of hypertension, cardiovascular disease, renal dysfunction, edema, cerebrovascular disease, and insulinopathies.

In another embodiment the pathological condition is selected from the group consisting of hypertension, cardiovascular disease, stroke, and Type II diabetes mellitus.

In another embodiment the pathological condition is selected from the group consisting of hypertension, heart failure (particularly heart failure post myocardial infarction), left ventricular hypertrophy, and stroke.

In another embodiment the pathological condition is hypertension.

In another embodiment the pathological condition is heart failure.

In another embodiment the pathological condition is myocardial infarction.

In another embodiment the pathological condition is stroke.

In another embodiment the pathological condition is atherosclerosis.

In another embodiment the pathological condition is renal dysfunction.

In another embodiment the pathological condition is organ damage.

In another embodiment the pathological condition is diabetes.

Subjects in Need of Treatment or Prevention

In addition to being suitable for human use, the present combination therapy is also suitable for treatment of animals, including mammals such as horses, dogs, cats, rats, mice, sheep, pigs, and the like.

In one embodiment the subject is a human exhibiting one or more of the following characteristics:

(a) The average daily intake of sodium chloride by the subject is at least about 4 grams, particularly where this condition is satisfied over any one month interval for at least one or more monthly intervals over a given annual period. The average daily intake of sodium by the subject is at least about 6 grams. In another embodiment, the average daily intake of sodium by the subject is at least about 8 grams. In still another embodiment, the average daily intake of sodium by the subject is at least about 12 grams.

(b) The subject exhibits an increase in systolic blood pressure and/or diastolic blood pressure of at least about 5%, when daily sodium chloride intake by the subject is increased from less than about 3 g/day to at least about 10 g/day. In another embodiment, the subject exhibits an increase in systolic blood pressure and/or diastolic blood pressure of at least about 7%. In still another embodiment, the subject exhibits an increase in systolic blood pressure and/or diastolic blood pressure of at least about 10%.

(c) The activities ratio of plasma aldosterone (ng/dL) to plasma renin (ng/ml/hr) in the subject is greater than about 30. In another embodiment, the activities ratio is greater than about 40. In another embodiment, the activities ratio is greater than about 50. In still another embodiment, the activities ratio is greater than about 60.

(d) The subject has low plasma renin levels; for example, the morning plasma renin activity in the subject is less than about 1.0 ng/dL/hr, and/or the active renin value in the subject is less than about 15 pg/mL.

(e) The subject suffers from or is susceptible to elevated systolic and/or diastolic blood pressure. In one embodiment, the systolic blood pressure (measured, for example, by seated cuff mercury sphygmomanometer) of the subject is at least about 130 mm Hg. In another embodiment, the systolic blood pressure is at least about 140 mm Hg. In still another embodiment, the systolic blood pressure is at least about 150 mm Hg. Examples of elevated diastolic blood pressure (measured, for example, by seated cuff mercury sphygmomanometer) include at least about 85 mm Hg, at least about 90 mm Hg, and at least about 100 mm Hg.

(f) The urinary sodium to potassium ratio (mmol/mmol) of the subject is less than about 6; less than about 5.5; less than about 5; or less than about 4.5.

(g) The urinary sodium level of the subject is at least 60 mmol per day, particularly where this condition is satisfied over any one month interval for at least one or more monthly intervals over a given annual period. In another embodiment, the urinary sodium level of the subject is at least about 100 mmol per day. In another embodiment, at least about 150 mmol per day. In still another embodiment, at least about 200 mmol per day.

(h) The plasma concentration of one or more endothelins, particularly plasma immunoreactive ET-1, in the subject is elevated. Examples include plasma concentrations of ET-1 greater than about 2.0 pmol/L, greater than about 4.0 pmol/L, and greater than about 8.0 pmol/L.

(i) The subject has blood pressure that is substantially refractory to treatment with an ACE inhibitor; examples include a subject whose blood pressure is lowered less than about 8 mm Hg, less than 5 mm Hg, and less than 3 mm Hg, in response to 10 mg/day enalapril compared to the blood pressure of the subject on no antihypertensive therapy.

(j) The subject has blood volume-expanded hypertension or blood volume-expanded borderline hypertension, that is, hypertension wherein increased blood volume as a result of increased sodium retention contributes to blood pressure.

(k) The subject is a non-modulating individual, that is, the individual demonstrates a blunted positive response in renal blood flow rate and/or in adrenal production of aldosterone to an elevation in sodium intake or to angiotensin II administration, particularly when the response is less than the response of individuals sampled from the general geographical population (for example, individuals sampled from the subject's country of origin or from a country of which the subject is a resident). Examples include when the response is less than 40% of the mean of the population; when the response is less than 30%; and when the response is still less than 20%.

(l) The subject has or is susceptible to renal dysfunction, particularly renal dysfunction selected from one or more members of the group consisting of reduced glomerular filtration rate, microalbuminuria, and proteinuria.

(m) The subject has or is susceptible to cardiovascular disease, particularly cardiovascular disease selected from one or more members of the group consisting of heart failure, left ventricular diastolic dysfunction, hypertrophic cardiomyopathy, and diastolic heart failure.

(n) The subject has or is susceptible to liver disease, particularly liver cirrhosis.

(o) The subject has or is susceptible to edema, particularly edema selected from one or more members of the group consisting of peripheral tissue edema, hepatic or splenic congestion, liver ascites, and respiratory or lung congestion.

(p) The subject has or is susceptible to insulin resistance, particularly Type I or Type II diabetes mellitus, and/or glucose sensitivity.

(q) In one embodiment, the subject is, in whole or in part, a member of at least one ethnic group selected from the Asian (particularly from the Japanese) ethnic group, the American Indian ethnic group, and the African ethnic group.

(r) The subject has one or more genetic markers associated with salt sensitivity.

(s) The subject is obese. Examples include subjects having greater than 25% body fat; greater than 30% body fat; and greater than 35% body fat.

(t) The subject has one or more 1^{st}, 2^{nd}, or 3^{rd} degree relatives who are or were salt sensitive, wherein 1^{st} degree relatives means parents or relatives sharing one or more of the same parents, 2^{nd} degree relatives means grandparents and relatives sharing one or more of the same grandparents, and 3^{rd} degree relatives means great-grandparents and relatives sharing one or more of the same great-grandparents. In one embodiment, individuals who have four or more salt sensitive 1^{st}, 2^{nd}, or 3^{rd} degree relatives. In another embodiment, eight or more such relatives. In another embodiment, 16 or more such relatives. In still another embodiment, individuals who have 32 or more such relatives.

Unless otherwise indicated to the contrary, the values listed above represent an average value. In another embodiment, the values listed above represent a daily average value based on at least two measurements.

In one embodiment, the subject in need of treatment satisfies at least two or more of the above-characteristics. In another embodiment, the subject in need of treatment satisfies at least three or more of the above-characteristics. In still another embodiment, the subject in need of treatment satisfies at least four or more of the above-characteristics.

Accordingly, in one embodiment of the present invention the subject in need of treatment is salt sensitive and satisfies two or more of the following conditions: (i) the average daily intake of sodium chloride by the subject is at least about 4 grams, particularly where this condition is satisfied over any one month interval for at least one or more monthly intervals over a given annual period; and/or (ii) the activities ratio of plasma aldosterone (ng/dL) to plasma renin (ng/ml/hr) in the subject is greater than about 30; (iii) the morning plasma renin activity in the subject is less than about 1.0 ng/dL/hr, and/or the active renin value in the subject is less than about 15 pg/mL; and/or (iv) the systolic blood pressure of the subject is at least about 130 mm Hg and the diastolic blood pressure of the subject is at least about 85 mm Hg; and/or (v) the subject has or is susceptible to cardiovascular disease, particularly cardiovascular disease selected from one or more members of the group consisting of heart failure, left ventricular diastolic dysfunction, hypertrophic cardiomyopathy, and diastolic heart failure. In another embodiment of the present invention, the subject in need of treatment is salt sensitive and satisfies the following conditions: (i) the activities ratio of plasma aldosterone (ng/dL) to plasma renin (ng/ml/hr) in the subject is greater than about 30; and (ii) the morning plasma renin activity in the subject is less than about 1.0 ng/dL/hr, and/or the active renin value in the subject is less than about 15 pg/mL.

In another embodiment of the present invention, the subject in need of treatment is salt sensitive and satisfies at least two of the following conditions: (i) the average daily intake of sodium chloride by the subject is at least about 4 grams, particularly where this condition is satisfied over any one month interval for at least one or more monthly intervals over a given annual period; and/or (ii) the systolic blood pressure of the subject is at least about 130 mm Hg and the diastolic blood pressure of the subject is at least about 85 mm Hg; and/or (iii) the subject has or is susceptible to cardiovascular disease, particularly cardiovascular disease selected from one or more members of the group consisting of heart failure, left ventricular diastolic dysfunction, hypertrophic cardiomyopathy, ischemic heart disease, and diastolic heart failure.

Mechanism of Action

The selective aldosterone blocker, eplerenone, has been shown to effectively lower blood pressure in clinical and experimental settings. Clinical studies, for example, have demonstrated antihypertensive efficacy as a monotherapy or when co-administered with other agents in hypertensive patient populations with varying etiologies. Under normal physiologic conditions, activation of the RAAS is regulated, in part, by a negative feedback loop which reduces RAAS activation in response to elevated activity of the system. Treatment with eplerenone interrupts this feedback loop resulting in dose-dependent elevation in plasma renin activity and aldosterone levels. Thus, combination therapies directed at counterbalancing activation of the RAAS and accompanying vasoconstrictive properties of angiotensin II resulting from the RAAS activation may offer a distinct advantage over eplerenone monotherapy. Combinations of renin inhibitors that reduce the formation of angiontensin I, and in turn, decrease the amount of angiotensin II are therefore likely to provide superior benefit beyond renin inhibitor and eplerenone monotherapy through complementary mechanisms.

Without being held to a specific mechanism of action for the present combination therapy, it is hypothesized that the administration of an aldosterone receptor antagonist in combination with a renin inhibitor is effective because of the distinct physiological effects and pathways of the drugs as well as the simultaneous and interrelated responses of these distinct classes of drugs on one or more target disorders. The combination therapy is further hypothesized to be effective because of the combined effect of these therapeutic agents on the biochemical feedback pathways that affects the regulation and release of aldosterone and other agents in the body. The interrelationship of Renin-Angiotensin-Aldosterone System, Neutral Endopeptidase System, and Kallikrein-Kinin System is illustrated in FIG. 1.

For purposes of illustration, in treating hypertension, aldosterone receptor antagonists block aldosterone from promoting the retention of sodium in the body. Blocking of aldosterone reduces fluid retention and lowers blood pressure levels. Renin inhibitors block renin from forming angiotensin I in the body. In addition to promoting vasodialation, administration of a renin inhibitor also can promote the release of aldosterone in the body to counter the resulting down regulation of the RAAS.

By administering an aldosterone receptor antagonist in combination with a renin inhibitor, further release of aldosterone is reduced inhibiting subsequent retention of fluids. As a result of the different pathways and the interrelationships of regulating aldosterone and other agents, the collective effect of these therapeutic compounds is potentially greater than additive.

Advantages of Combination Therapy

The co-administration of an aldosterone receptor antagonist and a renin inhibitor can potentially provide more than an additive benefit. For example, the hypertension-lowering effect resulting from the combination therapy methods described herein can be greater than the hypertension-lowering effect resulting from the monotherapeutic administration of each active agent alone. Where the effect is more than additive, a reduced amount of the aldosterone receptor antagonist and/or renin inhibitor is needed for combination therapy relative to monotherapy to achieve the desired result.

Accordingly, the combination therapy methods of this invention also can be used to treat or prevent a pathological condition wherein the combination therapy method results in reduced side effects than observed with the corresponding monotherapy to achieve a similar result. For example, reduction of the dose of the aldosterone receptor antagonist or renin inhibitor in the present combination therapy below the conventional monotherapeutic dose can minimize, or even eliminate, the side-effect profile that may be associated with monotherapeutic administration of the drug. In addition, combination therapy methods permit treatment or prevention of a pathological condition to be “fine-tuned” to treat the specific condition of a patient. Thus, by adjusting the dose of the aldosterone receptor antagonist and renin inhibitor, each compound is provided in a dose that matches the aldosterone and renin levels of an individual that need to be inhibited.

Other benefits of the present combination therapy can include, but are not limited to, the use of a selected group of aldosterone receptor antagonists and renin inhibitors, that provide a relatively quick onset of therapeutic effect and a relatively long duration of action. For example, a single dose of one of the selected antagonists or inhibitors may stay associated with the aldosterone receptors or inhibit renin for a longer period of time than if provided to a patient on a monotherapeutic basis.

Aldosterone Receptor Antagonists

The term “aldosterone receptor antagonist” denotes a compound capable of binding to an aldosterone receptor, as a competitive inhibitor of the action of aldosterone itself at the mineralocorticoid receptor site, so as to modulate the receptor-mediated activity of aldosterone.

The aldosterone receptor antagonists used in the methods of the present invention generally are spirolactone-type steroidal compounds. The term “spirolactone-type” is intended to characterize a structure comprising a lactone moiety attached to a steroid nucleus, typically at the steroid “D” ring, through a spiro bond configuration. A subclass of spirolactone-type aldosterone receptor antagonist compounds consists of epoxy-steroidal aldosterone receptor antagonist compounds such as eplerenone. Another subclass of spirolactone-type antagonist compounds consists of non-epoxy-steroidal aldosterone receptor antagonist compounds such as spironolactone.

The epoxy-steroidal aldosterone receptor antagonist compounds used in the method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties:

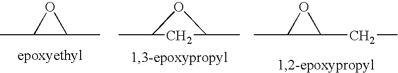

The term “steroidal,” as used in the phrase “epoxy-steroidal,” denotes a nucleus provided by a cyclopenteno-phenanthrene moiety, having the conventional “A,” “B,” “C,” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone receptor antagonists suitable for use in the present methods include a family of compounds having an epoxy moiety fused to the “C” ring of the steroidal nucleus. Examples include 20-spiroxane compounds characterized by the presence of a 9α, 11α-substituted epoxy moiety. Compounds 1 through 11, below, are illustrative 9α,11α-epoxy-steroidal compounds that may be used in the present methods. A particular benefit of using epoxy-steroidal aldosterone receptor antagonists, as exemplified by eplerenone, is the high selectivity of this group of aldosterone receptor antagonists for the mineralocorticoid receptor. The superior selectivity of eplerenone results in a reduction in side effects, that can be caused by aldosterone receptor antagonists that exhibit non-selective binding to non-mineralocorticoid receptors, such as androgen or progesterone receptors.

These epoxy steroids may be prepared by procedures described in Grob et al., U.S. Pat. No. 4,559,332. Additional processes for the preparation of 9, 11-epoxy steroidal compounds and their salts are disclosed in Ng et al., WO97/21720 and Ng et al., WO98/25948.

TABLE 1
9,11-Epoxy-Steroidal Aldosterone Receptor Antagonists

| Compound # | Structure | Name |
|---|---|---|
| A-1 | 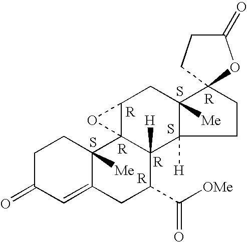 | Pregn-4-ene-7,21-dicarboxylic acid, 9,11-epoxy-17-hydroxy-3-oxo-,γ-lactone, methyl ester, (7α,11α,17β)- |
| A-2 | 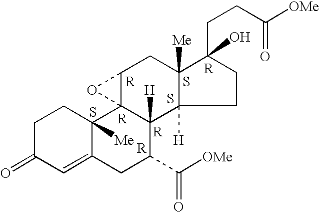 | Pregn-4-ene-7,21-dicarboxylic acid, 9,11-epoxy-17-hydroxy-3-oxo-,dimethyl ester, (7α,11α,17β)- |
| A-3 | 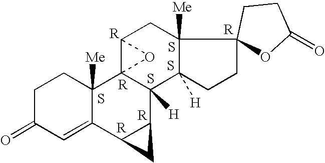 | 3′H-cyclopropa[6,7]pregna-4,6-diene-21- carboxylic acid,9,11-epoxy-6,7-dihydro- 17-hydroxy-3-oxo-,γ-lactone, (6β,7β,11α,17β)- |
| A-4 | 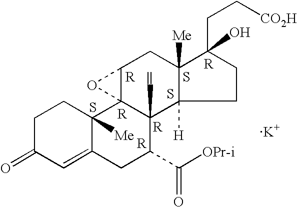 | Pregn-4-ene-7,21-dicarboxylic acid,9,11- epoxy-17-hydroxy-3-oxo-,7-(1-methylethyl) ester,monopotassium salt, (7α,11α,17β)- |
| A-5 | 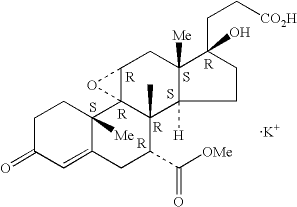 | Pregn-4-ene-7,21-dicarboxylic acid,9,11- epoxy-17-hydroxy-3-oxo-,7-methylethyl) ester,monopotassium salt, (7α,11α,17β)- |
| A-6 | 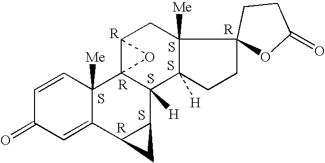 | 3′H-cyclopropa[6,7]pregna-1,4,6-triene- 21-carboxylic acid,9,11-epoxy-6,7-dihydro- 17-hydroxy-3-oxo-,Γ-lactone(6β,7β,11α)- |
| A-7 | 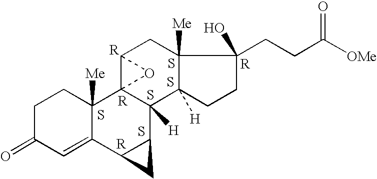 | 3′H-cyclopropa[6,7]pregna-4,6-diene-21- carboxylic acid, 9,11-epoxy-6,7-dihydro-17- hydroxy-3-oxo-, methyl ester, (6β,7β,11α,17β)- |
| A-8 | 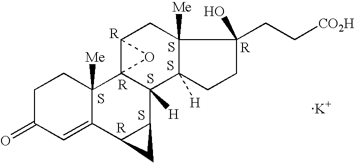 | 3′H-cyclopropa[6,7]pregna-4,6-diene-21- carboxylic acid, 9,11-epoxy-6,7-dihydro-17- hydroxy-3-oxo-, monopotassium salt, (6β,7β,11α,17β)- |
| A-9 | 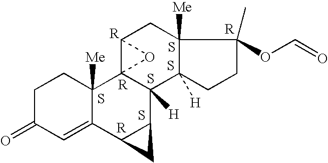 | 3′H-cyclopropa[6,7]pregna-1,4,6-triene-21- carboxylic acid, 9,11-epoxy-6,7-dihydro-17- hydroxy-3-oxo-,γ-lactone(6β,7β,11α,17β)- |
| A-10 | 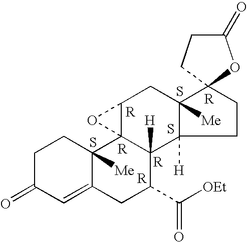 | Pregn-4-ene-7,21-dicarboxylic acid,9,11- epoxy-17-hydroxy-3-oxo-,γ-lactone,ethyl ester,(7α,11α,17β)- |
| A-11 | 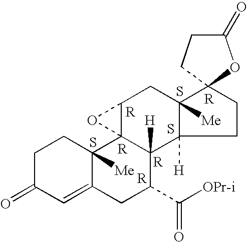 | Pregn-4-ene-7,21-dicarboxylic acid,9,11- epoxy-17-hydroxy-3-oxo-,γ-lactone,1- methylethyl ester (7α,11α,17β)- |

Of particular interest is the compound eplerenone (CAS No. 107724-20-9), also known as epoxymexrenone. Eplerenone is an aldosterone receptor antagonist and has a higher selectivity for aldosterone receptors than does, for example, spironolactone. Selection of eplerenone as the aldosterone receptor antagonist in the present method would be beneficial to reduce certain side-effects such as gynecomastia that occur with use of aldosterone receptor antagonists having less specificity.

Non-epoxy-steroidal aldosterone receptor antagonists suitable for use in the present methods include a family of spirolactone-type compounds defined by Formula I:

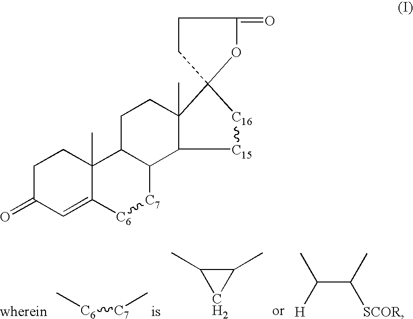

wherein R is lower alkyl of up to 5 carbon atoms, and

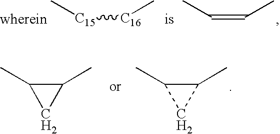

Lower alkyl residues include branched and unbranched groups, for example, methyl, ethyl and n-propyl.

Specific compounds of interest within Formula I are the following:

7α-acetylthio-3-oxo-4,15-androstadiene-[17(β-1′)-spiro-5′]perhydrofuran-2′-one;

3-oxo-7α-propionylthio-4,15-androstadiene-[17((β-1′)-spiro-5′]perhydrofuran-2′-one;

6β,7β-methylene-3-oxo4,15-androstadiene-[17((β-1′)-spiro-5′]perhydrofuran-2′-one;

15α,16α-methylene-3-oxo-4,7α-propionylthio-4-androstene[17(β-1′)-spiro-5′]perhydrofuran-2′-one;

6β,7β,15α,16α-dimethylene-3-oxo-4-androstene[17(β-1′)-spiro-5′]-perhydrofuran-2′-one;

7α-acetylthio-15β,16β-Methylene-3-oxo-4-androstene-[17(β-1′)-spiro-5′]perhydrofuran-2′-one;

15β,16β-methylene-3-oxo-7β-propionylthio-4-androstene-[17(β-1′)-spiro-5′]perhydrofuran-2′-one; and

6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on 12 Dec. 1978.

Another family of non-epoxy-steroidal compounds of interest is defined by Formula II:

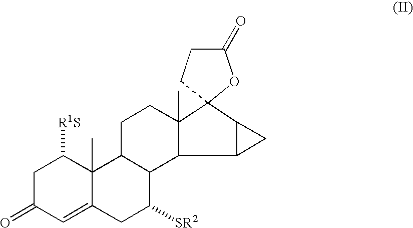

wherein R¹ is C₁₋₃-alkyl or C₁₋₃ acyl and R² is H or C₁₋₃-alkyl.

Specific compounds of interest within Formula II are the following:

1α-acetylthio-15β,16β-methylene-7α-methylthio-3-oxo-17α-pregn-4-ene-21,17-carbolactone; and

15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued 6 Dec. 1988.

Yet another family of non-epoxy-steroidal compounds of interest is defined by a structure of Formula III:

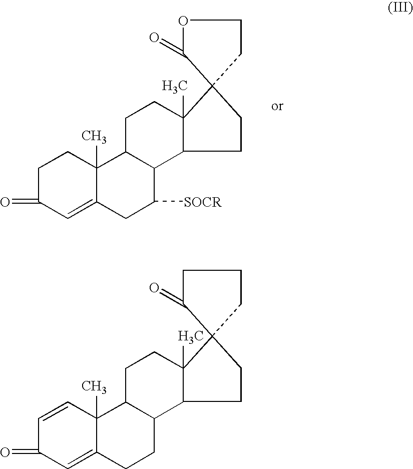

wherein R is lower alkyl, examples of which include lower alkyl groups of methyl, ethyl, propyl and butyl. Specific compounds of interest include:

3β,21-dihydroxy-17α-pregna-5,15-diene-17-carboxylic acid γ-lactone;

3β,21-dihydroxy-17α-pregna-5,15-diene-17-carboxylic acid γ-lactone 3-acetate;

3β,21-dihydroxy-17α-pregn-5-ene-17-carboxylic acid γ-lactone;

3β,21-dihydroxy-17α-pregn-5-ene-17-carboxylic acid γ-lactone 3-acetate;

21-hydroxy-3-oxo-17α-pregn-4-ene-17-carboxylic acid γ-lactone;

21-hydroxy-3-oxo-17α-pregna-4,6-diene-17-carboxylic acid γ-lactone;

21-hydroxy-3-oxo-17α-pregna-1,4-diene-17-carboxylic acid γ-lactone;

7α-acylthio-21-hydroxy-3-oxo-17α-pregn-4-ene-17-carboxylic acid γ-lactone; and

7α-acetylthio-21-hydroxy-3-oxo-17α-pregn-4-ene-17-carboxylic acid γ-lactone.

Methods to make the compounds of Formula III are described in U.S. Pat. No. 3,257,390 to Patchett which issued 21 Jun. 1966.

Still another family of non-epoxy-steroidal compounds of interest is represented by Formula IV:

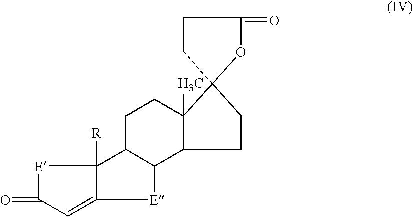

wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl)thio radical is present.

One family of non-epoxy-steroidal compounds within Formula IV is represented by Formula V:

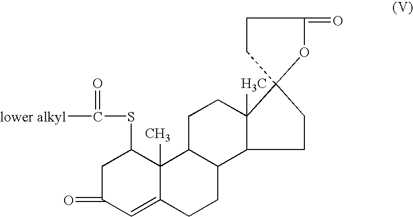

Another compound of Formula V is 1-acetylthio-17α-(2-carboxyethyl)-17β-hydroxy-androst-4-en-3-one lactone.

Another family of non-epoxy-steroidal compounds within Formula IV is represented by Formula VI:

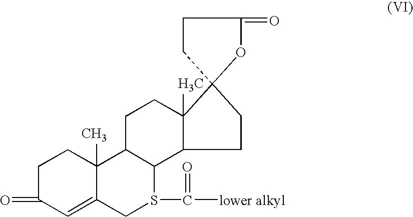

Exemplary compounds within Formula VI include the following:

7α-acetylthio-17α-(2-carboxyethyl)-17β-hydroxy-androst-4-en-3-one lactone;

7β-acetylthio-17α-(2-carboxyethyl)-17β-hydroxy-androst-4-en-3-one lactone;

1α,7α-diacetylthio-17α-(2-carboxyethyl)-17β-hydroxy-androsta-4,6-dien-3-one lactone;

7α-acetylthio-17αe-(2-carboxyethyl)-17β-hydroxy-androsta-1,4-dien-3-one lactone;

7α-acetylthio-17α-(2-carboxyethyl)-17β-hydroxy-19-norandrost-4-en-3-one lactone; and

7α-acetylthio-17α-(2-carboxyethyl)-17β-hydroxy-6α-methylandrost-4-en-3-one lactone.

In Formulae IV-VI, the term “alkyl” is intended to embrace linear and branched alkyl radicals containing one to about eight carbons. The term “(lower alkanoyl)thio” embraces radicals of the formula lower alkyl

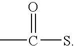

Of particular interest is the compound spironolactone having the following structure and formal name:

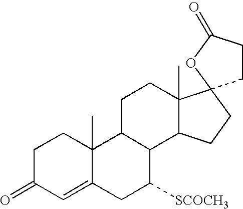

“spironolactone”: 17-hydroxy-7α-mercapto-3-oxo-17α-pregn-4-ene-21-carboxylic acid γ-lactone acetate.

Methods to make compounds of Formulae IV-VI are described in U.S. Pat. No. 3,013,012 to Cella et al. which issued 12 Dec. 1961. Spironolactone is sold by G. D. Searle & Co., Skokie, Ill., under the trademark “ALDACTONE”, in tablet dosage form at doses of 25 mg, 50 mg and 100 mg per tablet.

Another family of steroidal aldosterone receptor antagonists is exemplified by drospirenone, (6R-(6α, 7α, 8β, 9α, 10β, 13β, 14α, 15α, 16α, 17β))-1, 3′, 4′, 6, 7, 8, 9, 10, 11, 12, 13, 14, 15, 16, 20, 21-hexadecahydro-10, 13-dimethylspiro [17H-dicyclopropa(6,7:15,16)cyclopenta(a)phenanthrene-17,2′(5′H)-furan)-3,5′(2H)-dione, CAS registration number 67392-87-4. Methods to make and use drospirenone are described in patent GB 1550568 1979, priority DE 2652761 1976.

Crystalline forms that are easily handled, reproducible in form, easily prepared, stable, and which are non-hygroscopic have been identified for the aldosterone receptor antagonist eplerenone. These include Form H, Form L, various crystalline solvates and amorphous eplerenone. These forms, methods to make these forms, and use of these forms in preparing compositions and medicaments, are disclosed in Barton et al., WO 01/41535 and Barton et al., WO 01/42272 both incorporated herein in their entirety.

In one embodiment, Form H eplerenone may be administered in combination with a renin inhibitor. In another embodiment, Form L eplerenone may be administered in combination with a renin inhibitor. In another embodiment, a mixture of Form H and Form L eplerenone may be administered in combination with a renin inhibitor. In still another embodiment, the amorphous form of eplerenone may be administered in combination with a renin inhibitor.

Renin Inhibitors

Renin inhibitors, as described herein, encompass a wide range of structures and are useful in the combinations and methods of the present invention. Nonlimiting examples of renin inhibitors that may be used in the present invention are listed in Table 2, including the diastereomers, enantiomers, racemates, salts, esters, tautomers, conjugate acids, and prodrugs thereof. The renin inhibitor references identified in Table 2 are incorporated herein in their entirety.

In some instances, a renin inhibitor in its active form is not readily absorbed or bioavailable to the subject to whom it is being administered. If a prodrug form of the renin inhibitor is more readily absorbed or bioavailable, the prodrug form may be administered in place of the active form of the renin inhibitor.

In one embodiment, the combination therapy of the present invention comprises administering a first amount of an aldosterone receptor antagonist and a second amount of a renin inhibitor wherein (a) the renin inhibitor is selected from the group consisting of the renin inhibitors listed below in Table 2, including the diastereomers, enantiomers, racemates, salts, esters, tautomers, conjugate acids, and prodrugs thereof, and (b) the first amount of aldosterone receptor antagonist and second amount of renin inhibitor together comprise a therapeutically effective amount for the treatment or prevention of the pathological condition.

In another embodiment, the combination therapy of the present invention comprises administering a first amount of eplerenone and a second amount of a renin inhibitor wherein (a) the renin inhibitor is selected from the group consisting of the renin inhibitors listed below in Table 2, and (b) the first amount of eplerenone and second amount of renin inhibitor together comprise a therapeutically effective amount for the treatment or prevention of a pathological condition.

In another embodiment, the combination therapy of the present invention comprises administering a first amount of spironolactone and a second amount of a renin inhibitor wherein (a) the renin inhibitor is selected from the group consisting of the renin inhibitors listed below in Table 2, and (b) the first amount of spironolactone and second amount of renin inhibitor together comprise a therapeutically effective amount for the treatment or prevention of a pathological condition.

In another embodiment, the combination therapy of the present invention comprises administering a first amount of eplerenone and a second amount of a renin inhibitor selected from the group consisting of aliskiren, ditekiren, enalkiren, remikiren, terlakiren, and zankiren. The first amount of eplerenone and second amount of renin inhibitor together comprise a therapeutically effective amount for the treatment or prevention of the pathological condition.

In another embodiment, the combination therapy of the present invention comprises administering a first amount of spironolactone and a second amount of a renin inhibitor selected from the group consisting of aliskiren, ditekiren, enalkiren, remikiren, terlakiren, and zankiren. The first amount of spironolactone and second amount of renin inhibitor together comprise a therapeutically effective amount for the treatment or prevention of the pathological condition.

In one embodiment, the combination therapy of the present invention comprises administering a first amount of an aldosterone receptor antagonist and a second amount of a renin inhibitor wherein (a) the renin inhibitor is selected from the group consisting of aliskiren, ditekiren, enalkiren, remikiren, terlakiren, and zankiren, and (b) the first amount of aldosterone receptor antagonist and second amount of renin inhibitor together comprise a therapeutically effective amount for the treatment of a pathological condition, and (c) the first amount of aldosterone receptor antagonist produces no substantial diuretic and/or anti-hypertensive effect in the subject.

TABLE 2
Renin Inhibitors in Clinical Evaluation

| Generic | | | |
|---|---|---|---|
| Name(s) of | CAS* Registry Number | | |
| Renin | and | | Reference to Source |
| Inhibitor | Chemical Name | Chemical Structure | of Compound |
| aliskiren; CGP 60536; CGP60536B | 173334-57-1;Benzeneoctanamide, δ-amino-N-(3-amino-2,2-dimethyl- 3-oxopropyl)-γ-hydroxy-4- methoxy-3-(3-methoxy-propoxy)- α,ζ-bis(1-methylethyl)-, (αS,γS,δS,ζS)- | 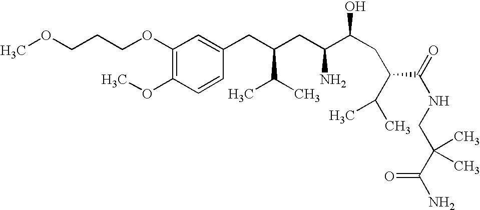 | U.S. Pat. No. 5,559,111; European Patent Application 678,503 Published: Oct. 25, 1995 |
| ditekiren; U 71038 | 103336-05-6; L-Isoleucinamide,1-[(1,1- dimethylethoxy) carbonyl]-L- prolyl-L-phenylalanyl-N-methyl- L-histidyl-(2S,4S,5S)-5-amino- 4-hydroxy-7-methyl-2-(1- methylethyl)octanoyl-N-(2- pyridinylmethyl)- | 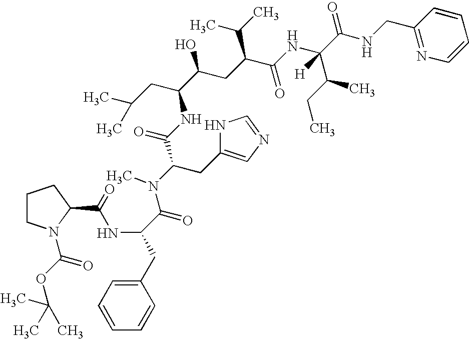 | European Patent Application 173,481 Published: Mar. 5, 1986 |
| enalkiren; A 64662; Abbott 64662 | 113082-98-7; L-Histidinamide,N-(3-amino-3- methyl-1-oxobutyl)-O-methyl-L- tyrosyl-N-[(1S,2R,3S)-1- (cyclohexyl-methyl)-2,3- dihydroxy-5-methylhexyl]- | 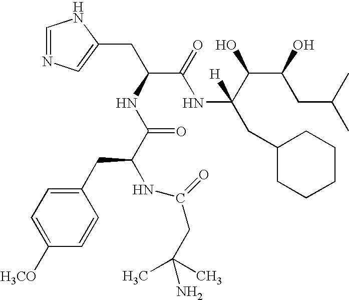 | European Patent Application 311,012 Published: Apr. 12, 1989 |
| remikiren; Ro 42-5892; | 126222-34-2; 1H-Imidazole-4-propanamide,N- [(1S,2R,3S)-1-(cyclohexyl- methyl)-3-cyclopropyl-2,3- dihydroxypropyl]-α-[[(2S)-2- [[(1,1-dimethylethyl)- sulfonyl]methyl]-1-oxo-3- phenylpropyl]amino]-,(αS)- | 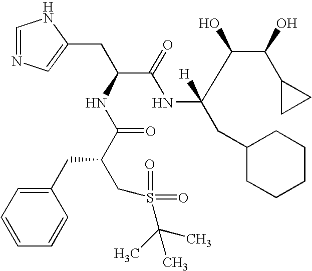 | European Patent Application 416,373 Published: Mar. 13, 1991 |
| terlakiren; CP 80794 | 119625-78-4; L-Cysteinamide,N-(4- morpholinylcarbonyl)-L- phenylalanyl-N-[(1S,2R)-1- (cyclohexylmethyl)-2-hydroxy-3- (1-methylethoxy)-3-oxopropyl]- s-methyl- | 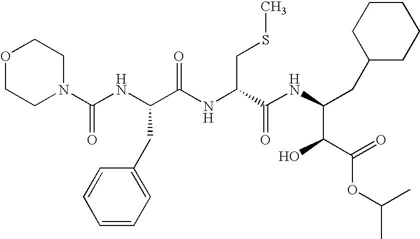 | European Patent Application 266,950 Published: May. 11, 1988 |
| zankiren; A 72517 | 138742-43-5; 4-Thiazolepropanamide,N- [(1S,2R,3S)-1-(cyclohexyl- methyl)-2,3-dihydroxy-5- methylhexyl]-α-[[(2S)-2-[[(4- methyl-1-piperazinyl)- sulfonyl]methyl]-1-oxo-3- phenylpropyl]amino]-,(αS)- | 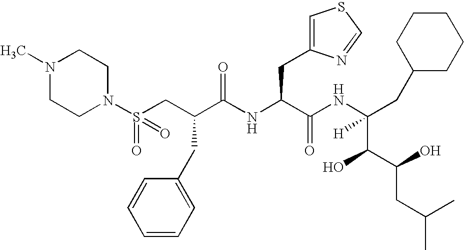 | European Patent Application 456,185 Published: Nov. 13, 1991 |

A combination therapy of the present invention may also comprise administering a first amount of an aldosterone receptor antagonist and a second amount of a renin inhibitor. The first amount of the aldosterone receptor inhibitor additionally exhibits a release profile, determined using a suitable release profile test, in which more than about 20% by weight of the aldosterone receptor antagonist is released at about four hours after initiation of the test.

In another embodiment, the first amount of the aldosterone receptor antagonist exhibits a release profile in which at least about 30% by weight of the aldosterone receptor antagonist is released at about four hours after initiation of the test.

In another embodiment, the first amount of the aldosterone receptor antagonist exhibits a release profile in which at least about 50% by weight of the aldosterone receptor antagonist is released at about four hours after initiation of the test.

In another embodiment, the first amount of the aldosterone receptor antagonist exhibits a release profile in which at least about 70% by weight of the aldosterone receptor antagonist is released from the composition at about four hours after initiation of the test.

In another embodiment, the combination therapy of the present invention comprises administering a first amount of eplerenone and a second amount of a renin inhibitor wherein the first amount of eplerenone and second amount of renin inhibitor together comprise a therapeutically effective amount for the treatment or prevention of a pathological condition. The first amount of eplerenone additionally exhibits a release profile, determined using a suitable release profile test, in which more than about 20% by weight of eplerenone is released at about four hours after initiation of the test.

In another embodiment, the first amount of the eplerenone exhibits a release profile in which at least about 30% by weight of eplerenone is released at about four hours after initiation of the test.

In another embodiment, the first amount of the eplerenone exhibits a release profile in which at least about 50% by weight of the eplerenone is released at about four hours after initiation of the test.

In another embodiment, the first amount of the eplerenone exhibits a release profile in which at least about 70% by weight of the eplerenone is released from the composition at about four hours after initiation of the test.

In another embodiment, the combination therapy of the present invention comprises administering a first amount of spironolactone and a second amount of a renin inhibitor, wherein the first amount of spironolactone and second amount of renin inhibitor together comprise a therapeutically effective amount for the treatment or prevention of a pathological condition. The first amount of spironolactone additionally exhibits a release profile, determined using a suitable release profile test, in which more than about 20% by weight of spironolactone is released at about four hours after initiation of the test.

In another embodiment, the first amount of the spironolactone exhibits a release profile in which at least about 30% by weight of spironolactone is released at about four hours after initiation of the test.

In another embodiment, the first amount of the spironolactone exhibits a release profile in which at least about 50% by weight of spironolactone is released at about four hours after initiation of the test.

In another embodiment, the first amount of the spironolactone exhibits a release profile in which at least about 70% by weight of spironolactone is released from the composition at about four hours after initiation of the test.

In another embodiment, the combination therapy of the present invention comprises administering a first amount of an aldosterone receptor antagonist and a second amount of a renin inhibitor wherein the first amount of aldosterone receptor antagonist and second amount of renin inhibitor together comprise a therapeutically effective amount for the treatment of a pathological condition. The first amount of aldosterone receptor antagonist produces no substantial diuretic and/or anti-hypertensive effect in the subject. The first amount of aldosterone receptor antagonist additionally exhibits a release profile, determined using a suitable release profile test, in which more than about 20% by weight of the aldosterone receptor antagonist is released at about four hours after initiation of the test.

Combinations and Compositions

The present invention is further directed to combinations, including pharmaceutical compositions, comprising one or more aldosterone receptor antagonists and one or more renin inhibitors. In one embodiment, the combination is a pharmaceutical composition comprising an aldosterone receptor antagonist, or a pharmaceutically acceptable salt, ester, or prodrug thereof; a renin inhibitor, or a pharmaceutically acceptable salt, ester, conjugate acid, or prodrug thereof; and a pharmaceutically acceptable carrier for treating pathological conditions. In one embodiment, the antagonist and inhibitor together comprise a therapeutically effective composition for treating a pathological condition. In another embodiment, the combination contains an amount of the aldosterone receptor antagonist that would not produce, if administered to a subject as a monotherapeutic dose without co-administration with other active agents, a substantial diuretic and/or blood pressure-lowering effect in the subject. Examples of aldosterone receptor antagonists and renin inhibitors used in the preparation of the compositions are as previously set forth above. The combinations and compositions comprising an aldosterone receptor antagonist and a renin inhibitor of the present invention can be administered for the treatment or prevention of a pathological condition, as previously set forth, by any means that produce contact of these compounds with their site of action in the body.

The combinations of the present invention also can be presented with an acceptable carrier in the form of a pharmaceutical composition. The carrier must be acceptable in the sense of being compatible with the other ingredients of the composition and must not be deleterious to the recipient. The carrier can be a solid or a liquid, or both, and in one embodiment is formulated with the compound as a unit-dose composition, for example, a tablet, which can contain from 0.05% to 95% by weight of the active compounds. Other pharmacologically active substances can also be present, including other compounds useful in the present invention. The pharmaceutical compositions of the invention can be prepared by any of the well-known techniques of pharmacy, such as admixing the components.

The combinations and compositions of the present invention can be administered by any-conventional means available for use in conjunction with pharmaceuticals. In one embodiment, the aldosterone receptor antagonist and the renin inhibitor is orally administered. The methods of the present invention are still effective when administered by other routes, for example, if the drugs are administered parenterally. The amount of each antagonist or inhibitor in the combination or composition that is required to achieve the desired biological effect will depend on a number of factors including those discussed below with respect to the treatment regimen.

Oral delivery of the aldosterone receptor antagonist and the renin inhibitor of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the inhibitor(s) and the carrier (which can constitute one or more accessory ingredients). In general, the compositions are prepared by uniformly and intimately admixing the antagonists and inhibitor(s) with a liquid or finely divided solid carrier, or both, and then, if necessary, shaping the product. For example, a tablet can be prepared by compressing or molding a powder or granules of the antagonists and inhibitors, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the inhibitors in an inert base such as gelatin and glycerin or sucrose and acacia.

In any case, the aldosterone receptor antagonist and renin inhibitor that can be combined with carrier materials to produce a single dosage form to be administered will vary depending upon the host treated and the particular mode of administration. The solid dosage forms for oral administration including capsules, tablets, pills, powders, and granules noted above comprise the inhibitors of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Pharmaceutically acceptable carriers encompass all the foregoing and the like. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

Triple or Multiple Combination Therapy

The present invention is further directed to combinations, including pharmaceutical compositions comprising an aldosterone receptor antagonist, a renin inhibitor, and one or more additional active drugs, and to the corresponding combination therapies whereby such multiple therapeutic agents are co-administered. Such compositions and combination therapies may be utilized for the treatment or prevention of the conditions previously discussed in this application Additional drugs co-administered with the aldosterone receptor antagonist and renin inhibitor can include, but are not limited to, for example, drugs selected from the group consisting of angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzyme inhibitors, vasodilators, cyclooxygenase-2 inhibitors, and diuretics.

Other drugs that can also be co-administered include, but are not limited to, members of the group consisting of lipid-lowering drugs (including apical sodium bile acid transport inhibitors, cholesterol absorption inhibitors, fibrates, niacin, statins, cholesteryl ester transfer protein inhibitors, and bile acid sequestrants), anti-oxidants (including vitamin E and probucol), and IIb/IIIa antagonists.

Angiotensin-II receptor antagonists that are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. Patents are incorporated herein by reference.

Angiotensin converting enzyme inhibitors that are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. Patents are incorporated herein by reference.

Alpha-adrenergic receptor blockers that are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may top prepared as disclosed in U.S. Pat. No. 3,669,968; and yohimbine, which may be isolated from natural sources according to methods well known to those skilled in the art The disclosures of all such U.S. Patents are incorporated herein by reference.

Beta-adrenergic receptor blockers that are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; bubridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,769; nadolol, which may be prepared as disclosed in U.S. Pat. No. 3,935, 267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. Patents are incorporated herein by reference.

Calcium channel blockers that are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. Patents are incorporated herein by reference.

Endothelin receptor antagonists that are within the scope of this invention include, but are not limited to: Bosentan, described in U.S. Pat. No. 5,883,254, Sitaxsentan, described in U.S. Pat. No. 5,594,021, Darusentan, described in WO 99/16446, and endothelin receptor antagonist compositions as disclosed in U.S. Pat. No. 6,162,927, U.S. Pat. No. 6,043,265, U.S. Pat. No. 5,952,327, U.S. Pat. No. 6,017,916, U.S. Pat. No. 6,107,320, U.S. Pat. No. 5,939,446, U.S. Pat. No. 5,922,681,

U.S. Pat. No. 6,197,821, U.S. Pat. No. 5,891,892,
U.S. Pat. No. 6,162,927, U.S. Pat. No. 6,124,341,
U.S. Pat. No. 5,846,985, U.S. Pat. No. 6,242,485,
U.S. Pat. No. 6,133,263, U.S. Pat. No. 6,297,274,
U.S. Pat. No. 5,658,943, U.S. Pat. No. 6,271,248,
U.S. Pat. No. 6,080,774, U.S. Pat. No. 5,998,468,
U.S. Pat. No. 5,612,359, U.S. Pat. No. 6,140,325,
U.S. Pat. No. 5,922,759, U.S. Pat. No. 6,017,951,
U.S. Pat. No. 6,258,817, U.S. Pat. No. 6,060,475,
U.S. Pat. No. 5,866,568, U.S. Pat. No. 5,576,439,
U.S. Pat. No. 5,739,333, U.S. Pat. No. 5,977,075,
U.S. Pat. No. 5,599,811, U.S. Pat. No. 5,760,038,
U.S. Pat. No. 6,004,965, U.S. Pat. No. 6,207,686,
U.S. Pat. No. 5,559,135, U.S. Pat. No. 5,514,696,
U.S. Pat. No. 5,538,991, U.S. Pat. No. 5,622,971,
U.S. Pat. No. 5,731,434, U.S. Pat. No. 5,767,144,
U.S. Pat. No. 5,550,110, U.S. Pat. No. 5,840,722,
U.S. Pat. No. 5,728,706, U.S. Pat. No. 5,693,637,
U.S. Pat. No. 5,420,123, U.S. Pat. No. 6,211,234,
U.S. Pat. No. 5,492,917, U.S. Pat. No. 5,714,479,
U.S. Pat. No. 5,389,620, U.S. Pat. No. 5,714,479,
U.S. Pat. No. 5,686,478, U.S. Pat. No. 5,391,566,
U.S. Pat. No. 5,888,972, U.S. Pat. No. 5,378,715,
U.S. Pat. No. 5,481,030, U.S. Pat. No. 5,420,133,
U.S. Pat. No. 5,374,638, U.S. Pat. No. 5,352,800,
U.S. Pat. No. 5,985,894, U.S. Pat. No. 5,550,138,
U.S. Pat. No. 5,550,138, U.S. Pat. No. 5,240,910,
U.S. Pat. No. 5,240,910, U.S. Pat. No. 5,616,684,
U.S. Pat. No. 5,883,075, U.S. Pat. No. 5,352,659,
U.S. Pat. No. 6,043,265, U.S. Pat. No. 6,043,265,
U.S. Pat. No. 5,780,473, U.S. Pat. No. 6,162,927,
U.S. Pat. No. 5,780,473, U.S. Pat. No. 6,124,343,
U.S. Pat. No. 6,048,893, U.S. Pat. No. 5,916,907,
U.S. Pat. No. 5,612,359, U.S. Pat. No. 5,565,485,
U.S. Pat. No. 5,641,793, U.S. Pat. No. 5,668,137,
U.S. Pat. No. 5,668,176, U.S. Pat. No. 5,691,373,
U.S. Pat. No. 5,767,310, U.S. Pat. No. 5,861,401,
U.S. Pat. No. 6,083,951, U.S. Pat. No. 5,866,568,
U.S. Pat. No. 6,017,916, U.S. Pat. No. 6,043,241,
U.S. Pat. No. 6,136,971, U.S. Pat. No. 6,218,427,
U.S. Pat. No. 6,251,861, U.S. Pat. No. 6,258,817,
U.S. Pat. No. 6,291,485, U.S. Pat. No. 6,297,274,
U.S. Pat. No. 5,846,990, and U.S. Pat. No. 5,795,909.

Endothelin converting enzyme inhibitors that are within the scope of this invention include, but are not limited to: endothelin receptor antagonist compositions which may be prepared as disclosed in U.S. Pat. No. 5,338,726, U.S. Pat. No. 5,380,921, U.S. Pat. No. 5,330,978, U.S. Pat. No. 35,886 (reissue), U.S. Pat. No. 5,952,327, and U.S. Pat. No. 5,550,119.

Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77 250 or synthesized as disclosed in Kennedy, Journal of Biological Chemistry, 1956, 222 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; ebumamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which maybe prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be preparedas disclosed in U.S. Pat. No. 3,334,096; nicametate, which may be prepared as disclosed in Blicke et al., Journal of the American Chemical Society, 1942 64 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. Patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which maybe prepared as disclosed in British Patent No. 807,826; droprenilamine, which maybe prepared as disclosed in German Patent No. 2,521,113; efloxate, which may be prepared as disclosed in British Patent Nos. 803,372 and 824,547; erythrityltetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflaznve, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which maybe prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. Patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which maybe prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947 69, 2907; isoxsuprine, which may be prepared as disclosed in U.S. Pat. No. 3,056,836; kallidin, which may be prepared as disclosed in Biochem. Biophys. Res. Commun.,1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin El, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinolniacinate, which may be prepared as disclosed in German Patent No. 1,102,750 or Korbonits et al., Acta. Pharm. Hung., 1968, 38, 98. The disclosures of all such U.S. Patents are incorporated herein by reference.

The term “diuretic”, within the scope of this invention, includes, but is not limited to, diuretic benzothiadiazine derivatives, diuretic organomercurials, diuretic purines, diuretic steroids (including diuretic steroids having no substantial activity as an aldosterone receptor antagonist), diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen, 1930, 478, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. Patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-0; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiaide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as disclosed in deStevens et al., Experientia, 1960, 16, 113. The disclosures of all such U.S. Patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosernide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared disclosed in Olivier, Rec. Trav. Chim., 1918, 37 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which maybe prepared, as disclosed in U.S. Pat. No. 3,567,777. The disclosures of all such U.S. Patents are incorporated herein by reference.

In another embodiment the aldosterone receptor antagonist and renin inhibitor can be administered in combination with an angiotensin I antagonist.

In another embodiment the aldosterone receptor antagonist and renin inhibitor can be administered in combination with an angiotensin II antagonist.

In another embodiment the aldosterone receptor antagonist and renin inhibitor can be administered in combination with an alpha-adrenergic receptor blocker.

In another embodiment the aldosterone receptor antagonist and renin inhibitor can be administered in combination with a beta-adrenergic receptor blocker.

In another embodiment the aldosterone receptor antagonist and renin inhibitor can be administered in combination with a calcium channel blocker.

In another embodiment the aldosterone receptor antagonist and renin inhibitor can be administered in combination with an endothelin receptor antagonist.

In another embodiment the aldosterone receptor antagonist and renin inhibitor can be administered in combination with an endothelin converting enzyme inhibitor.

In another embodiment the aldosterone receptor antagonist and renin inhibitor can be administered in combination with a vasodilator.

In another embodiment the aldosterone receptor antagonist and renin inhibitor can be administered in combination with a diuretic.

In another embodiment aldosterone receptor antagonist and renin inhibitor can be administered in combination with a member of the group consisting of lipid-lowering drugs (including apical sodium bile acid transport inhibitors, cholesterol absorption inhibitors, fibrates, niacin, statins, cholesteryl ester transfer protein inhibitors, and bile acid sequestrants).

In another embodiment the aldosterone receptor antagonist and renin inhibitor can be administered in combination with anti-oxidants (including vitamin E and probucol).

In another embodiment the aldosterone receptor antagonist and renin inhibitor can be administered in combination with a IIb/IIIa antagonist.

Administration of a aldosterone receptor antagonist and a renin inhibitor and optionally other therapeutic agents also can be effected in combination with one or more of non-drug therapies, such as non-drug therapies associated with the treatment of restenosis. For example, conventional treatment of restenosis resulting from angioplasty includes therapies such as exposing the artery at the site of injury to a source of radiation to inhibit restrictive neointima growth and inserting an endolumenal stent at the site of angioplasty.

In one embodiment, the aldosterone receptor antagonist and renin inhibitor can be administered in combination with exposure of an angioplastied artery at the site of injury to a source of radiation to inhibit restrictive neointima growth. Although radiation monotherapy has been used to prevent restenosis after angioplasty, Powers et al., Int. J. Radiat. Oncol. Biol, Vol. 45(3), pp. 753-759 (Oct. 1, 1999), report findings in a study involving a canine model that indicate that adventitial fibrosis increases with increasing dosages of radiation and can contribute to adverse late vascular remodeling. The proposed combination therapy would permit the use of dosages of radiation below conventional monotherapeutic dosages of radiation and would result in fewer side-effects or adverse effects relative to such radiation monotherapy.

In another embodiment, the stent itself comprises the aldosterone receptor antagonist and/or renin inhibitor and is used as a carrier to effect local delivery of the aldosterone receptor antagonist and/or renin inhibitor to the injured vessel. The aldosterone receptor antagonist and/or renin inhibitor is coated on, adsorbed on, affixed to or present on the surface of the stent or is otherwise present in or on the matrix of the stent, either alone or in combination with other active drugs and pharmaceutically acceptable carriers, adjuvants, binding agents and the like. In one embodiment, the stent comprises the aldosterone receptor antagonist and/or renin inhibitor in the form of an extended release composition that provides for release of the compound(s) over an extended period of time.

Aldosterone Receptor Antagonist/Renin Inhibitor Kits

The present invention further comprises kits comprising one or more aldosterone receptor antagonists and one or more renin inhibitors that are suitable for use in performing the methods of treatment and/or prevention described above. In one embodiment, the kit contains a first dosage form comprising one or more of the aldosterone receptor antagonists identified in Table 1 and a second dosage form comprising the renin inhibitors identified in Table 2 in quantities sufficient to carry out the methods of the present invention. The first dosage form and the second dosage form together comprise a therapeutically effective amount of the inhibitors for the treatment or prevention of a pathological condition.

In another embodiment, the kit contains a first dosage form comprising the aldosterone receptor antagonist spironolactone and a second dosage form comprising a renin inhibitor identified in Table 2 in quantities sufficient to carry out the methods of the present invention.

In another embodiment, the kit contains a first dosage form comprising the aldosterone receptor antagonist eplerenone and a second dosage form comprising a renin inhibitor identified in Table 2 in quantities sufficient to carry out the methods of the present invention.

In a further embodiment, the kit contains a first dosage form comprising the aldosterone receptor antagonist eplerenone, a second dosage form comprising a renin inhibitor identified in Table 2, and a third dosage of an active drug in quantities sufficient to carry out the methods of the present invention. Examples of active drugs which may be contained in the kit include, but are not limited to active drugs selected from the group consisting of angiotensin I antagonists, angiotensin II antagonists, angiotensin converting enzyme inhibitors, alpha-adrenergic receptor blockers, beta-adrenergic receptor blockers, calcium channel blockers, vasodilators, diuretics, cyclooxygenase-1 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists. Additionally, the active drug may be a second aldosterone receptor antagonist wherein the second aldosterone receptor antagonist produces no substantial diuretic and/or anti-hypertensive effect in a subject.

In another embodiment, the kit contains a first dosage form comprising one or more of the aldosterone receptor antagonists identified in Table 1 and a second dosage form comprising a renin inhibitor identified in Table 2 in quantities sufficient to carry out the methods of the present invention. The first dosage form and the second dosage form together comprise a therapeutically effective amount of the inhibitors for the treatment or prevention of a pathological condition. The first dosage form of the aldosterone receptor inhibitor additionally exhibits a release profile, determined using a suitable release profile test, in which more than about 20% by weight of the aldosterone receptor antagonist is released at about four hours after initiation of the test.

In another embodiment, the kit contains a first dosage form comprising the aldosterone receptor antagonist spironolactone and a second dosage form comprising a renin inhibitor identified in Table 2 in quantities sufficient to carry out the methods of the present invention. The first dosage form of the aldosterone receptor inhibitor additionally exhibits a release profile, determined using a suitable release profile test, in which more than about 20% by weight of the aldosterone receptor antagonist is released at about four hours after initiation of the test.

In another embodiment, the kit contains a first dosage form comprising the aldosterone receptor antagonist eplerenone and a second dosage form comprising a renin inhibitor identified in Table 2, in quantities sufficient to carry out the methods of the present invention. The first dosage form of the aldosterone receptor inhibitor additionally exhibits a release profile, determined using a suitable release profile test, in which more than about 20% by weight of the aldosterone receptor antagonist is released at about four hours after initiation of the test.

In another embodiment, the above-described kits may be comprised of an aldosterone receptor antagonist wherein the first dosage form of the aldosterone receptor inhibitor exhibits a release profile in which at least about 30% by weight of the aldosterone receptor antagonist is released at about four hours after initiation of the test.

In another embodiment, the above-described kits may be comprised of an aldosterone receptor antagonist wherein the first dosage form of the aldosterone receptor inhibitor exhibits a release profile in which at least about 50% by weight of the aldosterone receptor antagonist is released at about four hours after initiation of the test.

In still another embodiment, the above-described kits may be comprised of an aldosterone receptor antagonist wherein the first dosage form of the aldosterone receptor inhibitor exhibits a release profile in which at least about 70% by weight of the eplerenone is released from the composition at about four hours after initiation of the test.

Dosing Regimen

The dosing regimen to treat or prevent a pathological condition using the combinations and compositions of the present invention is selected in accordance with a variety of factors. These factors include the type, age, weight, sex, diet, and medical condition of the patient, the type and severity of the disease, the route of administration, pharmacological considerations such as the activity, efficacy, pharmacokinetics and toxicology profiles of the particular inhibitors employed, whether a drug delivery system is utilized, and whether the inhibitors are administered with other ingredients. Thus, the dosage regimen actually employed may vary widely and therefore deviate from the exemplary dosage regimen set forth above.

Initial treatment of a patient suffering from a pathological condition (such as hypertension, cardiovascular disease, renal dysfunction, edema, cerebrovascular disease, insulinopathies, and the like) can begin with the dosages indicated below. Treatment generally should be continued as necessary over a period of several weeks to several months or years until the pathological condition has been controlled or eliminated. Patients undergoing treatment with the combinations or compositions disclosed herein can be routinely monitored to determine treatment effectiveness. For example, in treating specific pathological conditions, measuring blood pressure, or other conventional indicators of the condition by any of the methods well-known in the art may be used to determine the effectiveness of the combination therapy. Continuous analysis of such data permits modification of the treatment regimen during therapy so that optimal effective amounts of each type of inhibitor are administered at any time, and so that the duration of treatment can be determined as well. In this way, the treatment regimen/dosing schedule can be rationally modified over the course of therapy so that the lowest amount of aldosterone receptor antagonist and renin inhibitor that together exhibit satisfactory effectiveness is administered, and so that administration is continued only so long as is necessary to successfully treat the pathological condition.

In combination therapy, administration of the aldosterone receptor antagonist and renin inhibitor may take place in sequence as part of a timed relationship in separate formulations, or may be accomplished by simultaneous administration in a single formulation or separate formulations.

When administered in a seqencence, the timed relationship between administration of the aldosterone receptor antagonist and renin inhibitor is less than 24 hours. In another embodiment the timed relationship is less than 12 hours. In another embodiment the timed relationship is less than 8 hours. In another embodiment the timed relationship is less than 6 hours. In another embodiment the timed relationship is less than 4 hours. In another embodiment the timed relationship is less than 1 hour. In another embodiment the timed relationship is less than thirty minutes. In another embodiment the timed relationship is less than ten minutes. In another embodiment the timed relationship is less than one minute.

Administration may be accomplished by any appropriate route, with oral administration being one embodiment. The dosage units used may with advantage contain one or more aldosterone receptor antagonists and one or more renin inhibitors in the amounts described below.

Dosing for oral administration may be with a regimen calling for a single daily dose, for multiple, spaced doses throughout the day, for a single dose every other day, for a single dose every several days, or other appropriate regimens. The aldosterone receptor antagonist and renin inhibitor used in the combination therapy may be administered simultaneously, either in a combined dosage form or in separate dosage forms intended for substantially simultaneous oral administration. The aldosterone receptor antagonist and renin inhibitor also may be administered sequentially, with antagonists and inhibitors being administered by a regimen calling for multiple-step ingestion. Thus, a regimen may call for sequential administration of the aldosterone receptor antagonist and renin inhibitor with spaced-apart ingestion of these separate, active agents. The time period between the multiple ingestion steps may range from a few minutes to several hours, depending upon the properties of each active agent such as potency, solubility, bioavailability, plasma half-life and kinetic profile of the inhibitor, as well as depending upon the age and condition of the patient. Dose timing may also depend on the circadian or other rhythms for the pathological effects of agents, such as aldosterone, which may be optimally blocked at the time of their peak concentration. The combination therapy, whether administration is simultaneous, substantially simultaneous, or sequential, may involve a regimen calling for administration of one therapeutic agent by oral route and another therapeutic agent by intravenous route. Whether these therapeutic agents are administered by oral or intravenous route, separately or together, each such therapeutic agent will be contained in a suitable pharmaceutical formulation of pharmaceutically acceptable excipients, diluents or other formulations components. Examples of suitable pharmaceutically acceptable formulations are given above.

Aldosterone Receptor Antagonist Dosing

The amount of aldosterone receptor antagonist that is administered and the dosage regimen for the methods of this invention depend on a variety of factors, including the age, weight, sex and medical condition of the subject, the severity of the pathological condition, the route and frequency of administration, and the particular aldosterone receptor antagonist employed, and thus may vary widely. A daily dose administered to a subject of between about 0.001 and about 30 mg/kg body weight, or between about 0.005 and about 20 mg/kg body weight, or between about 0.01 and about 15 mg/kg body weight, or between about 0.05 and about 10 mg/kg body weight, or between about 0.1 to 5 mg/kg body weight, may be appropriate. The amount of aldosterone receptor antagonist that is administered daily to a human subject typically will range from about 0.1 to 2000 mg, or from about 0.5 to 500 mg, or from about 0.75 to 250 mg, or from about 1 to 100 mg. A daily dose of aldosterone receptor antagonist that produces no substantial diuretic and/or anti-hypertensive effect in a subject is specifically embraced by the present method. The daily dose can be administered in one to six doses per day.

Where the aldosterone receptor antagonist is eplerenone, the daily dose administered typically is between about 10 mg to about 1000 mg. In one embodiment, the daily dose is between about 10 mg to about 400 mg. In another embodiment, the daily dose is between about 25 mg to about 200 mg. In still another embodiment, the daily dose is between about 50 mg to about 100 mg. Illustrative daily doses of eplerenone include, for example, 10, 20, 25, 37.5, 50, 75, 100, 125, 150, 175, 200, 250, 300, 350 or 400 mg of eplerenone.

Where the aldosterone receptor antagonist is spironolactone, the daily dose administered typically is between about 10 mg to about 1000 mg. In one embodiment, the daily dose is between about 10 mg to about 800 mg. In another embodiment, the daily dose is between about 25 mg to about 400 mg. In another embodiment, the daily dose is about 25 mg to about 200 mg. In still another embodiment, from about 50 mg to about 100 mg.

Dosing of the aldosterone receptor antagonist can be determined and adjusted based on measurement of blood pressure or appropriate surrogate markers (such as natriuretic peptides, endothelins, and other surrogate markers discussed below). Blood pressure and/or surrogate marker levels after administration of the aldosterone receptor antagonist can be compared against the corresponding baseline levels prior to administration of the aldosterone receptor antagonist to determine efficacy of the present method and titrated as needed. Non-limiting examples of surrogate markers useful in the method are surrogate markers for renal and cardiovascular disease.

Prophylactic Dosing

It can be beneficial to administer the aldosterone receptor antagonist prophylactically, prior to a diagnosis of pathological conditions, and to continue administration of the aldosterone receptor antagonist during the period of time the subject is susceptible to the pathological conditions. Individuals with no remarkable clinical presentation but that are nonetheless susceptible to pathological conditions therefore can be placed upon a prophylactic dose of an aldosterone receptor antagonist compound. Such prophylactic doses of the aldosterone receptor antagonist may, but need not, be lower than the doses used to treat the specific pathological condition of interest.

Renin Inhibitor Dosing

The amount of a renin inhibitor that is administered and the dosage regimen for the methods of this invention also depend on a variety of factors, including the age, weight, sex and medical condition of the subject, the severity of the pathological condition, the route and frequency of administration, and the particular renin inhibitor employed, and thus may vary widely. A renin inhibitor may be administered to a subject at a daily dose of about 0.001 to 100 mg/kg body weight, or between about 0.005 and about 60 mg/kg body weight, or between about 0.01 and about 50 mg/kg body weight, or between about 0.05 and about 30 mg/kg body weight, or between about 0.1 to 20 mg/kg body weight. For example, a daily dose of aliskiren that is typically administered to a subject is between about 0.003 mg/kg to about 0.3 mg/kg of body weight i.v., or between about 0.31 mg/kg to about 30 mg/kg of body weight p.o.

The amount of a renin inhibitor that is administered to a human subject will typically range from about 0.1 to 1000 mg, or from about 0.5 to 500 mg, or from about 0.75 to 250 mg, or from about 1.0 to 200 mg, or from about 5.0 to 100 mg, or from about 10.0 to 50 mg. The daily dose can be administered in one to six doses per day. For example, the amount of a aliskiren that is administered to a human subject typically will range from about 40 to about 640 mg per day, or from about 80 to 160 mg per day.

Cardiovascular Pathology Dosing

Dosing of the aldosterone receptor antagonist and renin inhibitor administered to treat cardiovascular-related conditions can be determined and adjusted based on measurement of blood concentrations of natriuretic peptides. Natriuretic peptides are a group of structurally similar but genetically distinct peptides that have diverse actions in cardiovascular, renal, and endocrine homeostasis. Atrial natriuretic peptide (“ANP”) and brain natriuretic peptide (“BNP”) are of myocardial cell origin and C-type natriuretic peptide (“CNP”) is of endothelial origin. ANP and BNP bind to the natriuretic peptide-A receptor (“NPR-A”), which, via 3′,5′-cyclic guanosine monophosphate (cGMP), mediates natriuresis, vasodilation, renin inhibition, antimitogenesis, and lusitropic properties. Elevated natriuretic peptide levels in the blood, particularly blood BNP levels, generally are observed in subjects under conditions of blood volume expansion and after vascular injury such as acute myocardial infarction and remain elevated for an extended period of time after the infarction. (Uusimaa et al.: Int. J. Cardiol 1999; 69: 5-14).

A decrease in natriuretic peptide level relative to the baseline level measured prior to administration of the aldosterone receptor antagonist indicates a decrease in the pathologic effect of aldosterone and therefore provides a correlation with inhibition of the pathologic effect. Blood levels of the desired natriuretic peptide level therefore can be compared against the corresponding baseline level prior to administration of the aldosterone receptor antagonist to determine efficacy of the present method in treating pathological conditions. Based upon such natriuretic peptide level measurements, dosing of the aldosterone receptor antagonist and renin inhibitor can be adjusted to reduce the cardiovascular pathological condition.

Similarly, cardiovascular-related conditions can also be identified, and the appropriate dosing determined, based on circulating and urinary cGMP Levels. An increased plasma level of cGMP parallels a fall in mean arterial pressure. Increased urinary excretion of cGMP is correlated with the natriuresis.

Cardiovascular-related conditions also can be identified by a reduced ejection fraction or the presence of myocardial infarction or heart failure or left ventricular hypertrophy. Left ventricular hypertrophy can be identified by echo-cardiogram or magnetic resonance imaging and used to monitor the progress of the treatment and appropriateness of the dosing.

In another embodiment of the invention, therefore, the methods of the present invention can be used to reduce natriuretic peptide levels, particularly BNP levels, thereby also treating related cardiovascular-related conditions.

Renal Pathology Dosing

Dosing of the aldosterone receptor antagonist and renin inhibitor administered to treat renal dysfunction can be determined and adjusted based on measurement of proteinuria, microalbuminuria, decreased glomerular filtration rate (GFR), or decreased creatinine clearance. Proteinuria is identified by the presence of greater than 0.3 g of urinary protein in a 24-hour urine collection. Microalbuminuria is identified by an increase in immunoassayable urinary albumin. Based upon such measurements, dosing of the aldosterone receptor antagonist and renin inhibitor can be adjusted to reduce the renal dysfunction.

Fixed Combination Dosage

Where the aldosterone receptor antagonist and the renin inhibitor are administered as a single dosage form, the ratio of aldosterone receptor antagonist to renin inhibitor (weight/weight) in that single dosage form typically will range from about 1:250 to about 250:1, or about 1:200 to about 200:1, or about 1:100 to about 100:1, or about 1:75 to about 75:1, or about 1:50 to about 50:1, or about 1:20 to about 20:1, or about 1:10 to about 10:1, or about 1:5 to about 5:1, or about 1:2 to about 2:1, or about 1:1.5 to about 1.5:1, or about 1:1.

Biological Evaluation

Human congestive heart failure (CHF) is a complex condition usually initiated by vascular hypertension or a myocardial infarction (MI). In order to determine the probable effectiveness of combination therapy for treating or preventing a cardiovascular-related condition, such as CHF, it is important to determine the potency of components in several assays. Accordingly, in Assays “A” and “B,” the activity of a renin inhibitor can be determined. In Assays “C” and “D,” a method is described for evaluating a combination therapy of the invention, namely, an aldosterone receptor antagonist or a combination of and an epoxy-steroidal aldosterone receptor antagonist and a renin inhibitor. The efficacy of individual drugs, such as eplerenone or a renin inhibitor, and the efficacy of these drugs given together at various doses, are evaluated in animal models of hypertension and CHF using surgical alterations to induce either hypertension or an MI. The methods of such assays are described below.

In addition, clinical trials can be used to evaluate aldosterone receptor antagonist therapy in humans. Numerous examples of such therapeutic tests have been published, including those of the RALES 003 study described in American Journal of Cardiology 78, 902-907 (1996); the RALES 004 study described in New England Journal of Medicine 341, 709-717 (1999); and the Eplerenone Post-Acute Myocardial Infarction Heart Failure Efficacy and Survival Study (EPHESUS), described in the New England Journal of Medicine 348, 1309-1321 (2003)

Assay A: In Vitro Vascular Smooth Muscle-Response

Thoracic aortas, removed from male Sprague-Dawley rats (350-550 g), are dissected free from surrounding connective tissue, and cut into ring segments each about 2-3 mm long. Smooth muscle rings are mounted for isometric tension recording in an organ bath filled with 10 mL of Krebs-Henseleit (K—H) solution, pH 7.4. This bathing solution is maintained at 37° C. and bubbled with 95% O₂/5% CO₂. The strips are stretched to a tension of 2 g and allowed to equilibrate. Isometric tension changes are monitored using an isometric transducer and recorded on a potentiometric recorder. A precontraction is produced by adding a catecholamine or by changing the solution to 30 mM K⁺. Contraction is maintained for 30 minutes, and the preparation washed with Krebs-Henseleit solution. After sixty minutes, contraction is induced in the same manner as described above. Subsequently, a solution containing natriuretic peptide, with or without different concentrations of a renin inhibitor, is added to obtain a concentration-response curve, measuring isometric tension and subsequently evaluating guanylyl cyclase activity of the thoracic aorta.

Assay B: In Vivo Intragastric Pressor Assay Response

Male Sprague-Dawley rats weighing 225-300 grams are anesthetized with methohexital (30 mg/kg, i.p.) and catheters were implanted into the femoral artery and vein. The catheters are tunneled subcutaneously to exit dorsally, posterior to the head and between the scapulae. The catheters are filled with heparin (1000 units/ml of saline). The rats are returned to their cage and allowed regular rat chow and water ad libitum. After full recovery from surgery (3-4 days), rats are placed in Lucite holders and the arterial line is connected to a pressure transducer. Arterial pressure is recorded on a Gould polygraph (mmHg). Epinephrine or norepinephrine is administered as a 30 ng/kg bolus via the venous catheter delivered in a 50 μl volume with a 0.2 ml saline flush. The pressor response in mm Hg is measured by the difference from pre-injection arterial pressure to the maximum pressure achieved. The catecholamine injection is repeated every 10 minutes until three consecutive injections yield responses within 4 mmHg of each other. These three responses are then averaged and represent the control response to catecholamines. The renin inhibitor compound is suspended in 0.5% methylcellulose in water and is administered by gavage. The volume administered is 2 ml/kg body weight. Catecholamine bolus injections are given at 30, 45, 60, 75, 120, 150, and 180 minutes after gavage. The pressor response to the catecholamine is measured at each time point. The rats are then returned to their cage for future testing. A minimum of 3 days is allowed between tests. Percent inhibition is calculated for each time point following gavage by the following formula: ((Control Response—Response at time point)/Control Response)×100.

Assay “C”: Hypertensive Rat Model

Male rats are made hypertensive by placing a silver clip with an aperture of 240 microns on the left renal artery, leaving the contralateral kidney untouched. Sham controls undergo the same procedure but without attachment of the clip. One week prior to the surgery, animals to be made hypertensive are divided into separate groups and drug treatment is begun. Groups of animals are administered vehicle, aldosterone receptor antagonist alone, renin inhibitor alone, and combinations of renin inhibitor and aldosterone receptor antogonist at various doses, an example of which is described in Table 8 below:

TABLE 8

| | | Combination of | |
|---|---|---|---|
| | Aldosterone | | Aldosterone |
| | Receptor | | Receptor |
| RENIN Inhibitor | Antagonist | Renin Inhibitor | Antagonist |
| (mg/kg/day) | (mg/kg/day) | (mg/kg/day) | (mg/kg/day) |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |
| 50 | 5 | 50 | 5 |
| | 20 | 50 | 20 |
| | 50 | 50 | 50 |
| | 100 | 50 | 100 |
| | 200 | 50 | 200 |

After 12 to 24 weeks, systolic and diastolic blood pressure, left ventricular end diastolic pressure, left vebtrucykar dP/dt, plasma and urinary cGMP, and heart rate are evaluated. The hearts are removed, weighed, measured and fixed in formalin. Collagen content of heart sections are evaluated using computerized image analysis of picrosirius stained sections. It is expected that rats treated with a combination therapy of renin inhibitor and aldosterone receptor antagonist components, as compared to rats treated with individual components alone, will show improvements in cardiac performance.

:Assay “D”: Myocardial Infarction Rat Model

Male rats are anesthetized and the heart is exteriorized following a left-sided thoracotaomy. The left anterior descending coronary artery is ligated with a suture. The thorax is closed and the animal recovers. Sham animals have the suture passed through without ligation. One-week prior to the surgery, animals to undergo infarction are divided into separate groups and drug treatment is begun. Typically, the combination therapy is initiated within 24 hours of myocardial infarction. Groups of animals are administered vehicle, aldosterone receptor antagonist alone, a renin inhibitor alone, or combinations of an aldosterone receptor antagonist and a renin inhibitor, at various doses, an example of which is described in Table 9 below:

TABLE 9

Combination of

| | Aldosterone | | Aldosterone |
|---|---|---|---|
| | Receptor | | Receptor |
| Renin Inhibitor | Antagonist | Renin Inhibitor | Antagonist |
| (mg/kg/day) | (mg/kg/day) | (mg/kg/day) | (mg/kg/day) |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |
| 50 | 5 | 50 | 5 |
| | 20 | 50 | 20 |
| | 50 | 50 | 50 |
| | 100 | 50 | 100 |
| | 200 | 50 | 200 |

After six weeks, systolic and diastolic blood pressure, left ventricular end diastolic pressure, left ventricular dP/dt, plasma and urinary cGMP, and heart rate are evaluated. The hearts are removed, weighed, measured and fixed in formalin. Collagen content of heart sections are evaluated using computerized image analysis of picrosirius stained sections. It is expected that rats treated with a combination therapy of renin inhibitor and aldosterone receptor antagonist components, as compared to rats treated with individual components alone, will show improvements in cardiac performance.

Human Clinical Therapy Protocols

### EXAMPLE A-1

### Comparison Study of the Efficacy and Safety of Eplerenone and a Renin Inhibitor Alone and in Combination With Each Other in Patients With Left Ventricular Hypertrophy and Essential Hypertension

A clinical study is conducted to evaluate the effect of a renin inhibitor and eplerenone, given alone or in combination with each other, on change in blood pressure (BP) and on change in left ventricular mass (LVM) as measured by magnetic resonance imaging (MRI) in patients with left ventricular hypertrophy (LVH) and with essential hypertension. The study is a multicenter, randomized, double-blind, placebo run-in, parallel group trial involving a minimum of 150 patients with LVH and essential hypertension and consisting of a one- to two-week pretreatment screening period followed by a two-week single-blind placebo run-in period and a nine-month double-blind treatment period.

Patients who will enter the single-blind placebo run-in period (1) will have a prior electrocardiogram that shows LVH (a) by the Sokolow Lyon voltage criteria (Sokolow M et al. Am Heart J 1949;37:161), or (b) by the Devereux criteria (LVMI=134 g/m² for males and =110 g/m² for females; see Neaton J D et al. JAMA 1993;27:713-724); and (2) will have a seated blood pressure as follows: seDBP=85 mmHg and <114 mmHg and seSBP>140 mmHg and =200 mmHg if not currently treated with antihypertensive medication.

During the single-blind placebo run-in period at Visit 2, all patients must have an echocardiogram that demonstrates LVH per the Devereux criteria. After completing the two-week single-blind placebo run-in period, and after an MRI has been received, and approved as acceptable by the core laboratory, patients will be randomized to one of three treatment groups: eplerenone, renin inhibitor, or eplerenone plus renin inhibitor 10 mg. For the first two weeks of double-blind treatment patients will receive (1) eplerenone 50 mg plus placebo, (2) renin inhibitor 50 mg plus placebo, or (3) eplerenone 50 mg plus renin inhibitor 50 mg. The dose of study medication will be force-titrated for all patients at Week 2 to (1) eplerenone 100 mg plus placebo, (2) renin inhibitor 100 mg plus placebo, or (3) eplerenone 100 mg plus renin inhibitor 100 mg. At Week 4 the dose of study medication will be force-titrated for all patients to (1) eplerenone 200 mg plus placebo, (2) renin inhibitor 150 mg plus placebo, or (3) eplerenone 200 mg plus renin inhibitor 150 mg). Table A-1A illustrates the above-described dosing scheme.

TABLE A-1A
Study Medication Dose Levels

| | Randomized Study Medication | | | |
|---|---|---|---|---|
| | | | Eplerenone + | Number of |
| Dose | | Renin | Renin | Tablets/ |
| Levels | Eplerenone | inhibitor | inhibitor | Capsules |
| Placebo | Placebo | Placebo | Placebo | 1 tablet/1 |
| Run-In | | | | capsule |
| Dose 1 | 50 mg | 50 mg | (50 + 50) mg | 1 tablet/1 |
| | | | | capsule |
| Dose 2 | 100 mg | 100 mg | (100 + 100) mg | 1 tablet/1 |
| | | | | capsule |
| Dose 3 | 200 mg | 150 mg | (200 + 150) mg | 2 tablets/ |
| | | | | 2 capsules |

If BP is not controlled (DBP=90 mmHg or SBP>180 mmHg) at Week 8, open-label hydrochlorothiazide (HCTZ) 12.5 mg will be added. If BP is uncontrolled at Week 10, (1) the HCTZ dose will be increased to 25 mg if HCTZ was started at Week 8, or (2) HCTZ 12.5 mg will be added if not done so at Week 8. If BP is not controlled at Week 12, (1) open-label HCTZ 12.5 mg will be added if not previously done so at Weeks 8 or 10, or (2) the HCTZ dose will be increased to 25 mg if not done so at Week 10. If at Week 16 or at any subsequent visit, the patient exhibits sustained uncontrolled DBP (i.e., seDBP=90 mmHg or seSBP>180 mmHg which persists at two consecutive visits, 3-10 days apart), the patient will be withdrawn from study participation.

If a patient is taking double-blind treatment alone and experiences symptomatic hypotension at any time during the trial, the patient will be withdrawn. Those patients taking open-label medications will have the open-label medications down-titrated in the reverse sequence as they were added until hypotension is resolved. If after all open-label medications are discontinued symptomatic hypotension is still present, the patient will be withdrawn from the trial. At any time during the study, if serum potassium level is elevated (>5.5 mEq/L) on repeat measurement at two consecutive visits 1-3 days apart, the patient will be withdrawn.

Patients will return to the clinic for evaluations at Weeks 0, 2, 4, 6, 8, 10, 12, 16, and monthly thereafter for a total of nine months. Heart rate, BP, serum potassium levels, and adverse events will be assessed at each visit. BUN and creatinine levels will be determined at Weeks 2 and 6. Additional laboratory assessments of blood for clinical safety will be done monthly. Routine urinalysis will be done every three months. A neurohormone profile (plasma renin (total and active), serum aldosterone, and plasma cortisol) and special studies (PIIINP, PAI, microalbuminuria, and tPA) will be done at Weeks 0, 12, and at Months 6 and 9. A blood sample for genotyping will be collected at Week 0. At screening and at Month 9, a 12-lead ECG and physical examination will be done. An MRI to assess changes in LV mass, a blood sample for storage retention, a blood sample for thyroid stimulating hormone (TSH), and a 24-hour urine collection for albumin, potassium, sodium, and creatinine will be done at Week 0 and at Month 9. A 24-hour urine collection for urinary aldosterone will be done at Weeks 0, 12 and at Months 6 and 9. In case of early termination, an MRI and blood sample for TSH will be done for those patients who have received double-blind treatment for at least three months.

The primary measure of efficacy is the change from baseline in LVM, as assessed by MRI, in the eplerenone group versus the renin inhibitor group versus the combination therapy group.

Secondary measures of efficacy will be the following: (1) the change from baseline in LVM among the three treatment groups; (2) the change from baseline of seated trough cuff DBP (seDBP) and SBP (seSBP) in each of the three treatment groups; (3) aortic compliance and ventricular filling parameters; and (4) special studies (PIIINP, microalbuminuria, PAI, and tPA). Additionally, the long-term safety and tolerability of the three treatment groups will be compared.

The primary objective of the study is to compare the effect of renin inhibitor versus eplerenone versus combination therapy, on change in left ventricular mass (LVM) in patients with LVH and with essential hypertension. The secondary objectives of the study are the following: (1) to compare the change from baseline in LVM among the three treatment groups; (2) to compare the antihypertensive effect among the three treatment groups as measured by seated trough cuff DBP and SBP; (3) to compare the effect of the three treatment groups on aortic compliance and ventricular filling parameters as measured by MRI; (4) to compare the effect of the three treatment groups on plasma markers of fibrosis by measuring the aminoterminal propeptide of Type III procollagen (PIIINP), on renal glomerular function by measuring microalbuminuria, and on fibrinolytic balance by measuring plasminogen activator inhibitor (PAI) and tissue plasminogen activator (tPA); and (5) to compare the long-term safety and tolerability of the three treatment groups.

Subgroup analyses of the primary and secondary efficacy measures can be performed with respect to other subgroups based on, for example, baseline recordings of such factors as gender, ethnic origin, age, plasma renin levels, aldosterone/renin activities ratio, urinary sodium to potassium ratio, presence of diabetes, history of hypertension, history of heart failure, history of renal dysfunction, and the like.

### EXAMPLE A-2

### Comparison Study of the Antihypertensive, Renal, and Metabolic Effects of Eplerenone Versus Renin Inhibitor Versus Combination Therapy, in Patients with Type 2 Diabetes Mellitus, Albuminuria, and Hypertension

A clinical study is conducted to compare the antihypertensive, renal, and metabolic effects of eplerenone alone, renin inhibitor alone, and the combination, in patients with Type 2 diabetes mellitus, albuminuria, and hypertension. The study is a multicenter, randomized, double-blind, active-controlled, placebo run-in, parallel group trial involving a minimum of 200 randomized patients with Type 2 diabetes mellitus, albuminuria, and hypertension. Each patient will be tested for salt sensitivity by salt challenge-unidirectional testing. The trial will further consist of a one- to two-week pretreatment screening period followed by a two- to four-week single-blind placebo run-in period and a 24-week double-blind treatment period. After completing the single-blind placebo run-in period, eligible patients will be randomized to one of three groups: eplerenone plus placebo, renin inhibitor plus placebo, or eplerenone plus renin inhibitor. For the first two weeks of double-blind treatment patients will receive eplerenone 50 mg plus placebo, renin inhibitor 50 mg plus placebo, or eplerenone 50 mg plus renin inhibitor 50 mg. At Week 2, the study medication dose will be force titrated to eplerenone 100 mg plus placebo, renin inhibitor 100 mg plus placebo, or eplerenone 100 mg plus renin inhibitor 100 mg. At Week 4, the dose will be force titrated to eplerenone 200 mg plus placebo, renin inhibitor 150 mg plus placebo, or eplerenone 200 mg plus renin inhibitor 150 mg. Table A-2A illustrates the above-described dosing scheme.

TABLE A-2A
Study Medication Dose Levels

| | Randomized Study Medication | | | |
|---|---|---|---|---|
| | | | Eplerenone + | Number of |
| Dose | | Renin | Renin | Tablets/ |
| Levels | Eplerenone | inhibitor | inhibitor | Capsules |
| Placebo | Placebo | Placebo | Placebo | 1 tablet/ |
| Run-In | | | | 1 capsule |
| Dose 1 | 50 mg | 50 mg | (50 + 50) mg | 1 tablet/ |
| | | | | 1 capsule |
| Dose 2 | 100 mg | 100 mg | (100 + 100) mg | 1 tablet/ |
| | | | | 1 capsule |
| Dose 3 | 200 mg | 150 mg | (200 + 150) mg | 2 tablets/ |
| | | | | 2 capsules |

If BP is not controlled (DBP=90 mmHg or SBP>180 mmHg) at Week 8, open-label hydrochlorothiazide (HCTZ) 12.5 mg will be added. If BP is uncontrolled at Week 10, (1) the HCTZ dose will be increased to 25 mg if HCTZ was started at Week 8, or (2) HCTZ 12.5 mg will be added if not done so at Week 8. If BP is not controlled at Week 12, (1) open-label HCTZ 12.5 mg will be added if not previously done so at Weeks 8 or 10, or (2) the HCTZ dose will be increased to 25 mg if not done so at Week 10. If at Week 15 or at any subsequent visit, the patient exhibits sustained uncontrolled DBP a95 mmHg which persists at two consecutive visits 3-10 days apart and the patient had received all add-on open-label medication as described above, the patient will be withdrawn from the study.

If symptomatic hypotension occurs and the patient is receiving open-label add-on medication, the add-on medication may be withdrawn in the reverse sequence as it was added until hypotension resolves. If the patient is not taking open-label medication, he/she must be withdrawn from the study.

At any time during the study, if DBP=110 or systolic BP [SBP]=180 mmHg persists at two consecutive visits 3-10 days apart, or if serum potassium level is elevated (>5.5 mEq/L) on repeat measurement, the patient will be withdrawn. Patients will return to the clinic for evaluations at Weeks 0, 2, 4, 6, 8, 10, 12, 15, 18, 21, 24, and 25. Heart rate, BP, body weight, serum potassium, and adverse events will be assessed at each visit. Hematology and biochemistry evaluations and urinalysis for safety will be at Weeks 0, 4, 6, 8, 10, 15, 21, 24, and 25. Collagen markers (aminoterminal propeptide of Type III procollagen [PIIINP], 7S domain of Type IV collagen [7SIVC], and Type I collagen telopeptide [ICTP]), fibrinolytic balance (plasminogen activator inhibitor [PAI-1] and tissue plasminogen activator [t-PA]), insulin, and glycosylated hemoglobin will be measured at Weeks 0, 8, 15, and 24. Albuminuria by 24-hour urine collection will be measured at Weeks 0, 8, and 24. A 12 lead electrocardiogram and physical examination will be done at screening and at Week 25. Genotype, waist circumference, plasma renin (total and active), and serum aldosterone will be measured at Week 0.

The primary measure of efficacy will be the change from baseline in urinary albumin excretion between eplerenone and renin inhibitor, or the combination, at Week 24. Additionally, efficacy will be evaluated with respect to the patient's degree of salt sensitivity by tertile (wherein tertiles are empirically determined by the increment of blood pressure response to salt challenge).

Secondary measures of efficacy will be the following: (1) the mean change from baseline in seated trough cuff DBP (“seDBP”) and SBP (seSBP) between eplerenone and renin inhibitor, or the combination, at Weeks 8 and 24; (2) the mean change from baseline in collagen markers (PIIINP, 7SIVC, and ICTP), fibrinolytic balance (PAI-1 and t-PA), and metabolic effects (insulin, glycosylated hemoglobin, fasting serum glucose, and lipids [triglycerides, total cholesterol, and HDL cholesterol]) between eplerenone and renin inhibitor, or the combination, at Week 24; (3) the mean change from baseline in antihypertensive, metabolic, or urinary albumin excretion response between eplerenone and renin inhibitor, or the combination, due to genotype, baseline truncal obesity, baseline plasma renin level (total and active), or baseline serum aldosterone level; and (4) Safety and tolerability will be assessed by adverse events, clinical laboratory values, physical examination, vital signs, and electrocardiogram.

This double-blind, active-controlled study is designed to determine the net effect of eplerenone on the insulin resistance, glycemic control, renal function, and lipid profile of hypertensive patients with NIDDM and albuminuria as compared to renin inhibitor or the combination. The primary objective of this study is to compare the mean change from baseline in urinary albumin excretion in patients treated with eplerenone versus enalapril or the combination at Week 24. The secondary objectives of this study are to (1) compare the effect on mean change from baseline of trough cuff seDBP and seSBP of eplerenone versus renin inhibitor or the combination at Weeks 8 and 24; (2) compare the effects of eplerenone versus renin inhibitor, or the combination, as measured by mean change from baseline of collagen markers (aminoterminal propeptide of Type III procollagen [PIIINP], 7S domain of Type IV collagen [7SIVC], and Type I collagen telopeptide [ICTP]); fibrinolytic balance (plasminogen activation inhibitor [PAI-1], tissue plasminogen activator [t-PA]), and metabolic effects (insulin, glycosylated hemoglobin, fasting serum glucose, and lipids [triglycerides, total cholesterol, and HDL cholesterol]) at Week 24; (3) measure any difference in mean change from baseline in antihypertensive, metabolic, or urinary albumin excretion response of eplerenone versus renin inhibitor or the combination due to genotype, baseline truncal obesity (waist circumference), baseline plasma renin level (total and active), or baseline serum aldosterone level; and compare the safety and tolerability of eplerenone versus renin inhibitor, or the combination, as assessed by reported adverse events, clinical laboratory values, physical examination, vital signs, and electrocardiogram.

Subgroup analyses of the primary and secondary efficacy measures can be performed with respect to other subgroups based on, for example, baseline recordings of such factors as ethnic background (black, non-black, Japanese, etc.), sex, age, plasma renin levels, aldosterone/renin activities ratio, urinary sodium to potassium ratio, history of heart failure, and the like.

### EXAMPLE A-3

### Dose-Ranging Study of Eplerenone and Renin Inhibitor, Alone and in Combination Vs. Placebo in Patients with Symptomatic Heart Failure

A clinical study is conducted to evaluate the safety and tolerability of a range of doses of eplerenone alone, renin inhibitor alone, and the combination, to assess their effect on neurohormonal function, and to examine their potential for improving signs and symptoms in patients with heart failure, optionally treated with an ACE inhibitor and/or a loop diuretic. Additionally, each of the above parameters will be evaluated with respect to the patient's degree of salt sensitivity by tertile (wherein tertiles are empirically determined by the increment of blood pressure response to salt challenge). The study is a randomized, double-blind, multicenter, placebo-controlled parallel group trial evaluating three different doses of eplerenone, a renin inhibitor, or the combination, vs. placebo. The study will enroll at least 400 patients. Each patient will be tested for salt sensitivity by salt challenge-unidirectional testing.

The study population will be patients with symptomatic heart failure who have an ejection fraction s 40% and are New York Heart Association (NYHA) Functional Class II-IV on entry. Patients eligible for the trial will receive one of the following treatments: renin inhibitor 100 mg QD; eplerenone 25 mg QD, 50 mg QD, 100 mg QD, with or without renin inhibitor 100 mg QD; or placebo. The measures for evaluation of neurohormones will be determinations of N-terminal atrial natriuretic peptide (N-terminal ANP), brain natriuretic peptide (BNP and pro-BNP), plasma renin (total and active), and plasma and urine aldosterone and cGMP. Assessment of patients′ signs and symptoms will be made using the NYHA Functional Class. Safety will be evaluated by the assessment of incidence of hyperkalemia and symptomatic hypotension, other adverse experiences, and clinical laboratory abnormalities. The study is structured to detect differences between eplerenone, eplerenone/renin inhibitor combination, and placebo treatment in the neurohormone levels and in major changes in clinical signs and symptoms.

The primary objectives of this study are (1) to evaluate the safety and tolerability of a range of doses of eplerenone, with or without co-administration of a renin inhibitor, in patients with HF; (2) to evaluate the effect of a range of doses of eplerenone, with or without co-administration of a renin inhibitor, on measurements of neurohormonal function [N-terminal atrial natriuretic peptide (ANP), brain natriuretic peptide (BNP) and its pro-form (pro-BNP), serum and urine aldosterone and cGMP, and plasma renin (total and active)] in patients with HF; and (3) to evaluate the efficacy of a range of doses of eplerenone, with or without co-administration of a renin inhibitor, given over 12 weeks in improving the signs and symptoms of HF as assessed by change from baseline in NYHA Functional Classification. The secondary objectives of this study are (1) to evaluate the effect of a range of doses of eplerenone co-administered with a renin inhibitor and optionally a loop diuretic and/or ACE inhibitor, on heart rate (HR), BP, and body weight; and (2) to evaluate the effect of eplerenone and eplerenone/renin inhibitor co-administration, on the changes in dosing of ACE inhibitors and diuretics when they are given concurrently with eplerenone or eplerenone/renin inhibitor combination.

If the patient becomes intolerant of study medication, alterations in the dose of concomitant medications (e.g., potassium supplements, ACE-I, etc.) should be considered prior to dose adjustment of study medication. If at any time during the study the serum potassium level equals 6.0 mEq/L, study medication is to be temporarily withheld. If serum potassium level is persistently equal to 6.0 mEq/L, the patient is to discontinue study medication. If elevated potassium levels are observed, potassium supplements, if any, should be stopped and the patient should continue to receive study medication. If study medication is stopped, concurrent medications should be reviewed and the doses adjusted if possible according to good clinical practice.

Subgroup analyses of the primary and secondary efficacy measures can be performed with respect to other subgroups based on, for example, baseline recordings of such factors as gender, ethnic origin, age, plasma renin levels, aldosterone/renin activities ratio, urinary sodium to potassium ratio, presence of diabetes, history of hypertension, history of renal dysfunction, and the like.

### EXAMPLE A-b 4

### Safety and Efficacy of Eplerenone and Eplerenone/Renin Inhibitor Combination Therapy, in Patients with Heart Failure Following Acute Myocardial Infarction

A clinical trial is conducted to compare the effect of eplerenone or eplerenone/renin inhibitor combination therapy, versus placebo on the rate of all cause mortality in patients with heart failure (HF) after an acute myocardial infarction (AMI). Secondary endpoints include cardiovascular morbidity and mortality. The study is a multicenter, randomized, double-blind, placebo-controlled, two-arm, parallel group trial will continue until 1,012 deaths occur, which is estimated to require approximately 6,200 randomized patients followed for an average of approximately 2.5 years. Patients eligible for this study will have (1) AMI (the index event) documented by (a) abnormal cardiac enzymes (creatine phosphokinase [CPK]>2× upper limit of the normal range [ULN] and/or CPK-MB>10% of total CPK), and (b) an evolving electrocardiogram (ECG) diagnostic of MI (progressive changes in ST segment and T wave compatible with AMI with or without presence of pathological Q waves); and (2) left ventricular (LV) dysfunction, demonstrated by LV ejection fraction (LVEF)=40% determined following AMI and before randomization; and (3) clinical evidence of HF documented by at least one of the following: (a) pulmonary edema (bilateral posttussive crackles extending at least ⅓ of the way up the lung fields in the absence of significant chronic pulmonary disease); or (b) chest x-ray showing pulmonary venous congestion with interstitial or alveolar edema; or (c) auscultatory evidence of a third heart sound (S₃) with persistent tachycardia (>100 beats per minute). Eligible patients may be identified for inclusion at any time following emergency room evaluation and presumptive diagnosis of AMI with HF. Patients who qualify for this study will be randomized between 3 (>48 hours) and 10 days post-AMI if their clinical status is stable, e.g., no vasopressors, inotropes, intra-aortic balloon pump, hypotension (systolic blood pressure [SBP]<90 mmHg), or recurrent chest pain likely to lead to acute coronary arteriography. Patients with implanted cardiac defibrillators are excluded.

Patients will be randomized to receive: eplerenone alone, 25 mg QD (once daily); renin inhibitor alone, 25 mg QD; combination therapy of eplerenone (25 mg QD) and renin inhibitor (25 mg QD); or placebo. At four weeks, the dose for all eplerenone dosed groups will be increased to 50 mg QD (two tablets) if serum potassium <5.0 mEq/L. If at any time during the study the serum potassium is >5.5 mEq/L but <6.0 mEq/L, the dose of eplerenone will be reduced to the next lower dose level, i.e., 50 mg QD to 25 mg QD (one tablet), 25 mg QD to 25 mg QOD (every other day), or 25 mg QOD to temporarily withheld. If at any time during the study the serum potassium is equal to 6.0 mEq/L, eplerenone should be temporarily withheld, and may be restarted at 25 mg QOD when serum potassium is <5.5 mEq/L. If at any time during the study the serum potassium is persistently equal to 6.0 mEq/L, study medications should be permanently discontinued. If the patient becomes intolerant of study medications, alterations in the dose of concomitant medications should be considered prior to dose adjustment of study medication. Serum potassium will be determined at 48 hours after initiation of treatment, at 1 and 5 weeks, at all other scheduled study visits, and within one week following any dose change.

Study visits will occur at screening, baseline (randomization), 1 and 4 weeks, 3 months, and every 3 months thereafter until the study is terminated. Medical history, cardiac enzymes, Killip class, time to reperfusion (if applicable), documentation of AMI and of HF, determination of LVEF, and a serum pregnancy test for women of childbearing potential will be done at screening. A physical examination and 12-lead ECG will be done at screening and at the final visit (cessation of study drug). Hematology and biochemistry evaluations and urinalysis for safety will be done at screening, Week 4, Months 3 and 6, and every 6 months thereafter until the study is terminated. An additional blood sample for DNA analysis will be collected during screening. Vital signs (seated heart rate and BP), New York Heart Association (NYHA) functional class, adverse events, and selected concurrent medications will be recorded at every visit. Quality of Life assessments will be completed during screening, at Week 4, Months 3, 6, and 12, and at the final visit. All randomized patients will be followed for endpoints every 3 months until the study is terminated.

The primary endpoint is all cause mortality. The trial is structured to detect an 18.5% reduction in all cause mortality, and requires 1,012 deaths before terminating the study. Secondary endpoints include (1) cardiovascular mortality; (2) sudden cardiac death; (3) death due to progressive heart failure; (4) all cause hospitalizations; (5) cardiovascular hospitalizations; (6) heart failure hospitalizations; (7) all cause mortality plus all cause hospitalizations; (8) cardiovascular mortality plus cardiovascular hospitalizations; (9) cardiovascular mortality plus heart failure hospitalizations; (10) new diagnosis of atrial fibrillation; (11) hospitalization for recurrent non-fatal AMI and fatal AMI; (12) hospitalization for stroke; and (13) quality of life.

Subgroup analyses of the primary and secondary endpoints will be performed. Subgroups will be based on baseline recordings of race (including black, non-black), gender, age, presence of diabetes, ejection fraction, serum potassium, serum creatinine, use of i-blockers, use of digoxin, use of potassium supplements, first versus subsequent AMI, Killip class, reperfusion status, history of hypertension, history of HF, history of smoking, history of angina, time from index AMI to randomization, and geographic region.

### EXAMPLE A-5

### Eplerenone, Compared to Eplerenone/Renin Inhibitor Co-Therapy, to Prevent or Treat Endothelial Dysfunction in Heart Failure Patients

Diagnosed heart failure patients (NYHA II-IV) will be pre-treated for two weeks with oral doses of one of the following: eplerenone (50 mg QD); renin inhibitor (50 mg QD); eplerenone(50 mg QD)/renin inhibitor (50 mg QD) co-therapy; or placebo. On test days, patients will be subjected to 20 minutes of supine rest, followed by cannulation of the nondominant brachial artery, under local anesthesia. After 30 minutes of saline infusion, baseline forearm blood flow is measured by forearm venous-occlusion plethysmography. Test solutions are then infused into the study arm with a constant rate infuser. Forearm blood flow is measured at each baseline and during the last two minutes of each test solution infusion. Blood pressure is measured in the non-infused (control) arm at regular intervals throughout the study.

Test solutions. First, acetylcholine (endothelium-dependant vasodilator) is infused at 25, 50, and 100 mmol/minute, each for five minutes. This is followed by sodium nitroprusside (endothelium independent vasodilator) at 4.2, 12.6, and 37.8 nmol/min, each for 5 minutes, and then N-monoethyl-L-arginine (L-NMMA; competitive NO synthase inhibitor) at 1, 2, and 4 μmol/min for 5 minutes each. This is followed by angiotensin I (vasoconstrictor only through conversion to angiotensin II) at 64, 256, and 1024 pmol/min for 7 minutes each. Between the different drugs, the drug infusion is flushed with saline for 20 to 30 minutes to allow sufficient time for the forearm blood flow to return to baseline values

Results. It is expected that, relative to placebo and other therapies, the combination therapy with eplerenone and renin inhibitor will significantly increase the forearm blood flow response to acetylcholine (percentage change in forearm blood flow), with an associated increase in vasoconstriction due to L-NMMA. It is further expected that the angiotensin I response will also be significantly reduced with combination therapy, while the response to angiotensin II remains unaltered. This study will further establish that heart failure is associated with endothelial dysfunction and decreased NO bioactivity. Furthermore, eplerenone/renin inhibitor co-therapy is expected to provide a superior benefit, relative to the other therapies tested, in preventing such dysfunction and related pathologic sequelae.

### EXAMPLE A-6

### Primary Prevention Events Trial in Dyslipidemic Patients

The following is a description of a clinical trial employing a co-therapy of an aldosterone receptor antagonist and a renin inhibitor to exemplify the methods of the present invention.

This is a primary prevention endpoint event trial. Inclusion criteria are LDL-cholesterol 130-190 mg/dl (or <130 if the ratio of total cholesterol/HDL is >6) and HDL-cholesterol <45 mg/dl. The trial is designed to study the effect of co-therapy of an aldosterone receptor antagonist and a renin inhibitor in a cohort with average to mildly elevated LDL-cholesterol and a below average HDL-cholesterol.

This is a double-blind, randomized, placebo controlled trial designed and powered to investigate whether co-therapy of an aldosterone receptor antagonist and a renin inhibitor will decrease the rate of first acute major coronary events (e.g., sudden cardiac death, fatal and non-fatal myocardial infarction and unstable angina) compared to intervention with eplerenone or a renin inhibitor alone. Secondary objectives include whether co-therapy treatment, compared to monotherapies, will decrease cardiovascular morbidity and mortality across the spectrum of clinical events, by measuring the rates of: (1) fatal and non-fatal coronary revascularization procedures (2) unstable angina, (3) fatal and non-fatal myocardial infarction, (4) fatal and non-fatal cardiovascular events, (5) fatal and non-fatal coronary events.

A four-week renin inhibitor alone baseline run-in is followed by randomization of participants to additional treatment with an aldosterone receptor antagonist, such as eplerenone, or placebo.

Baseline measurements at randomization include lipid analysis (including Apo A1 and Apo B), hematology, blood chemistry and urinalysis.

During the first year of active treatment, participants return to clinic at 4 week intervals. At each visit, participants are asked about adverse events and undergo laboratory safety tests for liver enzymes, creatine kinase and an extensive evaluation that includes a physical exam, electrocardiogram, mammography (women), ophthalmological examination, complete blood chemistry, hematology and urinalysis.

All subjects are followed until the decision to end the study after a median duration of 4 years of treatment. The trial design for the final analysis provides sufficient power to detect the reductions in the number of patients experiencing any of the following:

Primary Endpoints:

1—acute major coronary events defined as fatal and non-fatal myocardial infarction

2—unstable angina

3—sudden cardiac death

Secondary Endpoints:

1—revascularizations

2—unstable angina

3—fatal and nonfatal MI

4—fatal and nonfatal cardiovascular events

5—fatal and nonfatal coronary events

### EXAMPLE A-7

### Evaluation of Combination Therapy for Treatment of Coronary/Carotid artery Disease

The utility of the co-therapy of the present invention in treating atherosclerosis is demonstrated in the clinical trial protocol described below.

This study is a prospective double-blind, placebo-controlled trial of the effect of a combination of an aldosterone receptor antagonist and a renin inhibitor on the progression/regression of existing coronary artery disease as evidenced by changes in coronary angiography or carotid ultrasound.

Entry criteria: Subjects must be adult male or female, aged 18-80 years of age in whom coronary angiography is clinically indicated. Subjects will have angiographic presence of a significant focal lesion such as 30% to 50% on subsequent evaluation by quantitative coronary angiography (QCA) in a minimum of one segment. Segments to be analyzed include: left main, proximal, mid and distal left anterior descending, first and second diagonal branch, proximal and distal left circumflex, proximal, mid and distal right coronary artery.

At entry subjects undergo quantitative coronary angiography, B-mode carotid artery ultrasonography and assessment of carotid arterial compliance. Subjects are randomized to receive one of the following therapies: aldosterone receptor antagonist, renin inhibitor, co-therapy of an aldosterone receptor antagonist and a renin inhibitor, placebo. Subjects are monitored for three years. B-mode carotid ultrasound assessment of carotid artery atherosclerosis and compliance are performed at regular intervals throughout the study.

Coronary angiography is performed at the end of the three year period. Baseline and post-treatment angiograms and the intervening carotid artery B-mode ultrasonograms are evaluated for new lesions or progression of existing atherosclerotic lesions. Arterial compliance measurements are assessed for changes from baseline.

The primary objective of this study is to show that the co-therapy of an aldosterone receptor antagonist and a renin inhibitor, relative to placebo or monotherapies, reduces the progression of atherosclerotic lesions as measured by quantitative coronary angiography (QCA) in subjects with clinical coronary artery disease.

The primary endpoint of the study is the change in the average mean segment diameter of coronary arteries.

The secondary objective of this study is to demonstrate that the combination therapy, relative to placebo or monotherapies, reduces the rate of progression of atherosclerosis in the carotid arteries as measured by the slope of the maximum intimal-medial thickness measurements averaged over 12 separate wall segments (Mean Max) as a function of time.

In addition, numerous well-known, in vitro and in vivo testing schemes and protocols are useful to demonstrate the efficacy of aldosterone receptor antagonists and renin inhibitors, both separately and in combination, for treating or preventing pathological conditions. Non-limiting examples of testing schemes and protocols are described in references listed below, which are incorporated herein by reference.

Pitt, et al. NEJM 341, 709-717 (1999).

Pitt, et al. Cardiovasc Drug Ther 15:79-87 (2001).

De Gasparo, et al. J Pharm Exp Ther 240, 650-656 (1986).

Blazer-Yost, et al. Am. J. Physiol 272, C1928-C1935 (1997).

Vijan, et al. J Gen Intern Med 12, 567-580 (1997).

Gentile, et al. Diabetes, Obesity and Metabolism 2, 355-362 (2000).

Sheng-Fang, et al. Am J Cardiol 86, 514-518 (2000).

Jick, et al. Lancet 356, 1627-1631 (2000).

Albert, et al. JAMA 286, 64-70 (2001).

Ridker, et al. NEJM 344, 1959-1965 (2001).

Wang, et al. JAMA 283, 3211-3216 (2000).

Meier, et al. JAMA 283, 3205-3210 (2000).

Sugiyama, et al. Biochem Biophys Res Commun 271, 688-692 (2000).

Mundy, et al. Science 286, 1946-1949 (1999).

Xiao, et al. J Endocrinol 165, 533-536 (2000).

U.S. Pat. No. 5,730,992, U.S. Pat. No. 5,932,587, and U.S. Pat. No. 6,180,597; Huval et al., WO 00/69446; Huval et al., WO 00/69445; Cameron, WO 00/45818; Raza et al., WO 00/45817; Reszka, WO 99/66930; Scott, WO 99/11260; Delyani et al., WO 01/34132; and Alexander et al., WO 00/51642.

### COMPOSITION WORKING EXAMPLES

The following examples illustrate aspects of the present invention but should not be construed as limitations. The symbols and conventions used in these examples are consistent with those used in the contemporary pharmacological literature. Unless otherwise stated, (i) all percentages recited in these examples are weight percents based on total composition weight, (ii) total composition weight for capsules is the total capsule fill weight and does not include the weight of the actual capsule employed, and (iii) coated tablets are coated with a conventional coating material such as Opadry White YS-1-18027A and the weight fraction of the coating is about 3% of the total weight of the coated tablet.

### EXAMPLE B-1

An oral dosage may be prepared by screening and then mixing together the following list of ingredients in the amounts indicated. The dosage may then be placed in a hard gelatin capsule.

| Ingredients | Amounts |
|---|---|
| eplerenone | 12.5 mg |
| renin inhibitor | 12.5 mg |
| magnesium stearate | 10 mg |
| lactose | 100 mg |

### EXAMPLE B-2

An oral dosage may be prepared by mixing together granulating with a 10% gelatin solution. The wet granules are screened, dried, mixed with starch, talc and stearic acid, screened and compressed into a tablet.

| Ingredients | Amounts |
|---|---|
| eplerenone | 12.5 mg |
| renin inhibitor | 18 mg |
| calcium sulfate dihydrate | 100 mg |
| sucrose | 15 mg |
| starch | 8 mg |
| talc | 4 mg |
| stearic acid | 2 mg |

### EXAMPLE B-3

An oral dosage may be prepared by screening and then mixing together the following list of ingredients in the amounts indicated. The dosage may then be placed in a hard gelatin capsule.

| Ingredients | Amounts |
|---|---|
| eplerenone | 12.5 mg |
| renin inhibitor | 20 mg |
| magnesium stearate | 10 mg |
| lactose | 100 mg |

### EXAMPLE B-4

An oral dosage may be prepared by mixing together granulating with a 10% gelatin solution. The wet granules are screened, dried, mixed with starch, talc and stearic acid, screened and compressed into a tablet.

| Ingredients | Amounts |
|---|---|
| eplerenone | 12.5 mg |
| renin inhibitor | 30 mg |
| calcium sulfate dihydrate | 100 mg |
| sucrose | 15 mg |
| starch | 8 mg |
| talc | 4 mg |
| stearic acid | 2 mg |

### EXAMPLE B-5

### 25 Mg Dose Immediate Release Tablet

A 25 mg eplerenone dose immediate release tablet (tablet diameter of 7/32”) may be prepared having the following composition:

| | Amount |
|---|---|
| INGREDIENT | (mg) |
| Eplerenone | 25.00 |
| Renin Inhibitor | 25.00 |
| Lactose Monohydrate (#310, NF) | 35.70 |
| Microcrystalline Cellulose | 15.38 |
| (NF, Avicel PH101) | |
| Croscarmellose Sodium | 4.25 |
| (NE, Ac-Di-Sol) | |
| Hydroxypropyl Methylcellulose | 2.55 |
| (#2910, USP, Pharmacoat 603) | |
| Sodium Lauryl Sulfate (NF) | 0.85 |
| Talc (USP) | 0.85 |
| Magnesium Stearate (NF) | 0.42 |
| Total | 100 |
| Opadry White YS-1-18027A | 2.55 |

### EXAMPLE B-6

### 50 Mg Eplerenone Dose Immediate Release Tablet

A 50 mg eplerenone dose immediate release tablet (tablet diameter of 9/32”) may be prepared having the following composition:

| | Amount |
|---|---|
| INGREDIENT | (mg) |
| Eplerenone | 50.00 |
| Renin Inhibitor | 75.00 |
| Lactose Monohydrate (#310, NF) | 71.40 |
| Microcrystalline Cellulose | 30.75 |
| (NF, Avicel PH101) | |
| Croscarmellose Sodium | 8.50 |
| (NF, Ac-Di-Sol) | |
| Hydroxypropyl Methylcellulose | 5.10 |
| (#2910, USP, Pharmacoat 603) | |
| Sodium Lauryl Sulfate (NF) | 1.70 |
| Talc (USP) | 1.70 |
| Magnesium Stearate (NF) | 0.85 |
| Total | 235 |
| Opadry White YS-1-18027A | 5.10 |

### EXAMPLE B-7

100 Mg Eplerenone Dose Immediate Release Tablet

A 100 mg eplerenone dose immediate release tablet formulation (tablet diameter of 12/32”) was prepared having the following composition:

| | Amount |
|---|---|
| INGREDIENT | (mg) |
| Eplerenone | 100.00 |
| Renin Inhibitor | 10.00 |
| Lactose Monohydrate (#310, NF) | 142.80 |
| Microcrystalline Cellulose | 61.50 |
| (NF, Avicel PH101) | |
| Croscarmellose Sodium | 17.00 |
| (NF, Ac-Di-Sol) | |
| Hydroxypropyl Methylcellulose | 10.20 |
| (#2910, USP, Pharmacoat 603) | |
| Sodium Lauryl Sulfate (NF) | 3.40 |
| Talc (USP) | 3.40 |
| Magnesium Stearate (NF) | 1.70 |
| Total | 340 |
| Opadry White YS-1-18027A | 10.20 |

### EXAMPLE B-8

### 10 mg Eplerenone Dose Immediate Release Capsule

A 10 mg eplerenone dose immediate release capsule formulation was prepared having the following composition:

| | | REPRESENTATIVE |
|---|---|---|
| | AMOUNT | BATCH AMOUNT |
| INGREDIENT | (mg) | (kg) |
| Eplerenone | 10.0 | 1.00 |
| Renin Inhibitor | 10.0 | 1.00 |
| Lactose, Hydrous NF | 306.8 | 30.68 |
| Microcrystalline Cellulose, NF | 60.0 | 6.00 |
| Talc, USP | 10.0 | 1.00 |
| Croscarmellose Sodium, NF | 8.0 | 0.80 |
| Sodium Lauryl Sulfate, NF | 2.0 | 0.20 |
| Colloidal Silicon Dioxide, NF | 2.0 | 0.20 |
| Magnesium Stearate, NF | 1.2 | 0.12 |
| Total Capsule Fill Weight | 400.0 | 40.00 |
| Hard Gelatin Capsule, Size #0, | 1 Capsule | 100,000 Capsules |
| White Opaque | | |

### EXAMPLE B-9

### 25 mg Eplerenone Dose Immediate Release Capsule

A 25 mg eplerenone dose immediate release capsule formulation was prepared having the following composition:

| | | REPRESENTATIVE |
|---|---|---|
| | AMOUNT | BATCH AMOUNT |
| INGREDIENT | (mg) | (kg) |
| Eplerenone | 25.0 | 2.50 |
| Renin Inhibitor | 10.0 | 1.00 |
| Lactose, Hydrous NF | 294.1 | 29.41 |
| Microcrystalline Cellulose, NF | 57.7 | 5.77 |
| Talc, USP | 10.0 | 1.00 |
| Croscarmellose Sodium, NF | 8.0 | 0.80 |
| Sodium Lauryl Sulfate, NF | 2.0 | 0.20 |
| Colloidal Silicon Dioxide, NF | 2.0 | 0.20 |
| Magnesium Stearate, NF | 1.2 | 0.12 |
| Total Capsule Fill Weight | 400.0 | 40.00 |
| Hard Gelatin Capsule, Size #0, | 1 Capsule | 100,000 Capsules |
| White Opaque | | |

### EXAMPLE B-10

### 50 mg Eplerenone Dose Immediate Release Capsule

A 50 mg eplerenone dose immediate release capsule formulation was prepared having the following composition:

| | | REPRESENTATIVE |
|---|---|---|
| | AMOUNT | BATCH AMOUNT |
| INGREDIENT | (mg) | (kg) |
| Eplerenone | 50.0 | 5.00 |
| Renin Inhibitor | 10.0 | 1.00 |
| Lactose, Hydrous NF | 273.2 | 27.32 |
| Microcrystalline Cellulose, NF | 53.6 | 5.36 |
| Talc, USP | 10.0 | 1.00 |
| Croscarmellose Sodium, NF | 8.0 | 0.80 |
| Sodium Lauryl Sulfate, NF | 2.0 | 0.20 |
| Colloidal Silicon Dioxide, NF | 2.0 | 0.20 |
| Magnesium Stearate, NF | 1.2 | 0.12 |
| Total Capsule Fill Weight | 400.0 | 40.00 |
| Hard Gelatin Capsule, Size #0, | 1 Capsule | 100,000 Capsules |
| White Opaque | | |

### EXAMPLE B-11

### 100 mg Eplerenone Dose Immediate Release Capsule

A 100 mg Eplerenone dose immediate release capsule formulation was prepared having the following composition:

| | | REPRESENTATIVE |
|---|---|---|
| | AMOUNT | BATCH AMOUNT |
| INGREDIENT | (mg) | (kg) |
| Eplerenone | 100.0 | 10.00 |
| Renin Inhibitor | 10.0 | 1.00 |
| Lactose, Hydrous NF | 231.4 | 23.14 |
| Microcrystalline Cellulose, NF | 45.4 | 4.54 |
| Talc, USP | 10.0 | 1.00 |
| Croscarmellose Sodium, NF | 8.0 | 0.80 |
| Sodium Lauryl Sulfate, NF | 2.0 | 0.20 |
| Colloidal Silicon Dioxide, NF | 2.0 | 0.20 |
| Magnesium Stearate, NF | 1.2 | 0.12 |
| Total Capsule Fill Weight | 400.0 | 40.00 |
| Hard Gelatin Capsule, Size #0, | 1 Capsule | 100,000 Capsules |
| White Opaque | | |

### EXAMPLE B-12

### 200 mg Eplerenone Dose Immediate Release Capsule

A 200 mg eplerenone dose immediate release capsule formulation was prepared having the following composition:

| | | REPRESENTATIVE |
|---|---|---|
| | AMOUNT | BATCH AMOUNT |
| INGREDIENT | (mg) | (kg) |
| Eplerenone | 200.0 | 20.00 |
| Renin Inhibitor | 10.0 | 1.00 |
| Lactose, Hydrous NF | 147.8 | 14.78 |
| Microcrystalline Cellulose, NF | 29.0 | 2.90 |
| Talc, USP | 10.0 | 1.00 |
| Croscarmellose Sodium, NF | 8.0 | 0.80 |
| Sodium Lauryl Sulfate, NF | 2.0 | 0.20 |
| Colloidal Silicon Dioxide, NF | 2.0 | 0.20 |
| Magnesium Stearate, NF | 1.2 | 0.12 |
| Total Capsule Fill Weight | 400.0 | 40.00 |
| Hard Gelatin Capsule, Size #0, | 1 Capsule | 100,000 Capsules |
| White Opaque | | |

### Definitions

To facilitate understanding of the invention, a number of terms as used herein are defined below:

“Combination therapy” means the administration of two or more therapeutic agents to treat and/or prevent a pathological condition in a subject. Such administration encompasses co-administration of these therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. In either case, the treatment regimen will provide beneficial effects of the drug combination in treating the pathological condition.

“Epoxy-steroidal” is intended to embrace a steroidal nucleus having one or a plurality of epoxy-type moieties attached thereto.

“Pharmaceutically acceptable” is used adjectivally herein to mean that the modified noun is appropriate for use in a pharmaceutical product. Pharmaceutically acceptable cations include metallic ions and organic ions. Exemplary metallic ions include, but are not limited to, appropriate alkali metal salts, alkaline earth metal salts and other physiologically acceptable metal ions. Exemplary ions include aluminum, calcium, lithium, magnesium, potassium, sodium and zinc in their usual valences. Exemplary organic ions include protonated tertiary amines and quaternary ammonium cations, including in part, trimethylamine, diethylamine, N, N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

“Prodrug” refers to a chemical compound that is a drug precursor that, following administration to a subject and subsequent absorption, is converted to an active species in vivo via some process, such as metabolic conversion or simple chemical processes within the body of the subject. Other products from the conversion process are easily disposed of by the body. More preferred prodrugs produce products from the conversion process that are generally accepted as safe. For example, the prodrug may be an acylated form of the active compound.

“Renin Inhibitor” refers to any compound that can reduce or inhibit the activity of the enzyme renin. Further, these inhibitors reduce the formation of angiotensin I and angiotensin II, thus producing a hypotensive effect. A renin inhibitor includes prodrugs that are converted to an active renin inhibitor after they are within the body of a subject being administered the prodrug.

“Steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system.

“Subject” as used herein refers to an animal, a mammal, and particularly a human, who has been the object of treatment, observation or experiment.

“Therapeutically-effective” qualifies the amount of each agent that will achieve the goal of improvement in pathological condition severity and the frequency of incidence over treatment of each agent by itself, while avoiding adverse side effects typically associated with alternative therapies.

“Treatment” refers to providing any process, action, application, therapy, or the like to a subject, including a human being, for the cure, amelioration, or slowing the progression of a disease or pathological condition. “Treatment” further includes, but is not limited to, any process, action, application, therapy, or the like for preventing or inhibiting the development of the onset of a clinically evident pathological condition or a preclinically evident stage of a pathological condition in a subject. This term encompasses, but is not limited to, the prophylaxis, prevention, or inhibition of the development of a disease or pathological condition in a subject having a predisposition or possessing one or more risk factors for developing a disease or pathological condition such as, but not limited to, hypertension, cardiovascular disease, renal dysfunction, edema, cerebrovascular disease, and insulinopathy.

“Vasodilator” as used herein, is meant to include cerebral vasodilators, coronary vasodilators and peripheral vasodilators.

When introducing elements of the present invention or the embodiment(s) thereof, the articles “a”, “an”, and “the” are intended to mean that there are one or more of the elements. The terms “comprising”, “including” and “having” are intended to be inclusive and mean that there may be additional elements other than the listed elements.

We claim:

1. A method for the treatment of a pathological condition, the method comprising administering to a subject susceptible to or afflicted with such condition an aldosterone receptor antagonist and a renin inhibitor for the treatment of the pathological condition, wherein the renin inhibitor is selected from the group consisting of aliskiren, ditekiren, enalkiren, remikiren, terlakiren, and zankiren.

2. The method of claim 1, wherein the aldosterone receptor antagonist and renin inhibitor are administered simultaneously in a combined dosage form or in separate dosage forms.

3. The method of claim 1, wherein a first amount of the aldosterone receptor antagonist and a second amount of the renin inhibitor are provided to the subject in sequence as part of a timed relationship.

4. The method of claim 1, wherein the pathological condition is selected from the group consisting of hypertension, cardiovascular disease, renal dysfunction, liver disease, cerebrovascular disease, vascular disease, retinopathy, neuropathy, insulinopathy, edema, endothelial dysfunction, and baroreceptor dysfunction.

5. The method of claim 4,
wherein the cardiovascular disease is selected from the group consisting of heart failure, congestive heart failure, acute heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, and fibrinoid necrosis of coronary arteries;
wherein the renal dysfunction is selected from the group consisting of glomerulosclerosis, end-stage renal disease, acute renal failure, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis;
wherein the liver disease is selected from the group consisting of liver cirrhosis, liver ascites, and hepatic congestion;
wherein the cerebrovascular disease is stroke;
wherein the vascular disease is selected from the group consisting of thrombotic vascular disease, proliferative arteriopathy, atherosclerosis, decreased vascular compliance, and endothelial dysfunction;
wherein the insulinopathy is selected from the group consisting of insulin resistance, Type I diabetes mellitus, Type II diabetes mellitus, glucose resistance, pre-diabetic state, and syndrome X; and
wherein the edema is selected from the group consisting of peripheral tissue edema, hepatic congestion, splenic congestion, liver ascites, respiratory congestion, and lung congestion.

6. The method of claim 1, wherein the aldosterone receptor antagonist is an epoxy-steroidal-type compound characterized in having a 9α-,11α-substituted epoxy moiety.

7. The method of claim 1, wherein the aldosterone receptor antagonist is selected from the group consisting of eplerenone and spironolactone.

8. The method of claim 1, wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7)pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl)ester, monopotassium salt, (7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone (6α,7α,11α)-;
3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

9. The method of claim 1, wherein the aldosterone receptor antagonist is eplerenone.

10. The method of claim 1, wherein the aldosterone receptor antagonist is administered in a daily dose ranging from about 0.1 to 2000 mg, and the renin inhibitor is administered in a daily dose ranging from about 40 to 640 mg.

11. The method of claim 1, wherein the first amount of the aldosterone receptor antagonist produces no substantial diuretic or anti-hypertensive effect in a subject.

12. The method of claim 1, further comprising administering a third amount of a compound selected from the group consisting of angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

13. A method for the treatment of a pathological condition, the method comprising administering to a subject susceptible to or afflicted with such condition an aldosterone receptor antagonist and a renin inhibitor for the treatment of a pathological condition,
wherein the aldosterone receptor antagonist exhibits a release profile, determined using a suitable release profile test, in which more than about 20% by weight of the aldosterone receptor antagonist is released at about four hours after initiation of the test.

14. The method of claim 13, wherein the aldosterone receptor antagonist and renin inhibitor are administered simultaneously in a combined dosage form or in separate dosage forms.

15. The method of claim 13, wherein a first amount of the aldosterone receptor antagonist and a second amount of the renin inhibitor are provided to the subject in sequence as part of a timed relationship.

16. The method of claim 13, wherein the pathological condition is selected from the group consisting of hypertension, cardiovascular disease, renal dysfunction, liver disease, cerebrovascular disease, vascular disease, retinopathy, neuropathy, insulinopathy, edema, endothelial dysfunction, and baroreceptor dysfunction.

17. The method of claim 16,
wherein the cardiovascular disease is selected from the group consisting of heart failure, congestive heart failure, acute heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, and fibrinoid necrosis of coronary arteries;
wherein the renal dysfunction is selected from the group consisting of glomerulosclerosis, end-stage renal disease, acute renal failure, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis;
wherein the liver disease is selected from the group consisting of liver cirrhosis, liver ascites, and hepatic congestion;
wherein the cerebrovascular disease is stroke;
wherein the vascular disease is selected from the group consisting of thrombotic vascular disease, proliferative arteriopathy, atherosclerosis, decreased vascular compliance, and endothelial dysfunction;
wherein the insulinopathy is selected from the group consisting of insulin resistance, Type I diabetes mellitus, Type II diabetes mellitus, glucose resistance, pre-diabetic state, and syndrome X; and
wherein the edema is selected from the group consisting of peripheral tissue edema, hepatic congestion, splenic congestion, liver ascites, respiratory congestion, and lung congestion.

18. The method of claim 13, wherein the aldosterone receptor antagonist is an epoxy-steroidal-type compound characterized in having a 9α,11α-substituted epoxy moiety.

19. The method of claim 13, wherein the aldosterone receptor antagonist is selected from the group consisting of eplerenone and spironolactone.

20. The method of claim 13, wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl)ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone(6α,7α,11α)-;
3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

21. The method of claim 13, wherein the renin inhibitor is selected from the group consisting of aliskiren, ditekiren, enalkiren, remikiren, terlakiren and zankiren.

22. The method of claim 21, wherein the aldosterone receptor antagonist is eplerenone.

23. The method of claim 13, wherein the aldosterone receptor antagonist is administered in a daily dose ranging from about 0.1 to 2000 mg, and the renin inhibitor is administered in a daily dose ranging from about 40 to 640 mg.

24. The method of claim 13 wherein the first amount of the aldosterone receptor antagonist produces no substantial diuretic or anti-hypertensive effect in a subject.

25. The method of claim 13 further comprising administering a third amount of a compound selected from the group consisting of angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

26. A combination comprising an aldosterone receptor antagonist and a renin inhibitor in a pharmaceutically acceptable carrier, wherein the renin inhibitor is selected from the group consisting of aliskiren, ditekiren, enalkiren, remikiren, terlakiren, and zankiren.

27. The combination of claim 26 wherein the aldosterone receptor antagonist is selected from the group consisting of eplerenone and spironolactone.

28. A combination comprising an aldosterone receptor antagonist and a renin inhibitor in a pharmaceutically acceptable carrier,
wherein the aldosterone receptor antagonist exhibits a release profile, determined using a suitable release profile test, in which more than about 20% by weight of the aldosterone receptor antagonist is released at about four hours after initiation of the test.

29. The combination of claim 28 wherein the aldosterone receptor antagonist is selected from the group consisting of eplerenone and spironolactone.

30. A pharmaceutical composition comprising a first amount of an aldosterone receptor antagonist, a second amount of a renin inhibitor, and a pharmaceutically acceptable carrier.

31. The composition of claim 30, wherein the aldosterone receptor antagonist is an epoxy-steroidal-type compound characterized in having a 9α-,11α-substituted epoxy moiety.

32. The composition of claim 30, wherein the aldosterone receptor antagonist is selected from the group consisting of eplerenone and spironolactone.

33. The composition of claim 30, wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl)ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone(6α,7α,11α)-;
3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

34. The composition of claim 30, wherein the renin inhibitor is selected from the group consisting of aliskiren, ditekiren, enalkiren, remikiren, terlakiren, and zankiren.

35. The composition of claim 34, wherein the aldosterone receptor antagonist is eplerenone.

36. The composition of claim 35, wherein the composition contains between about 0.1 to about 200 mg of eplerenone.

37. The composition of claim 30, wherein the first amount of the aldosterone receptor antagonist produces no substantial diuretic or anti-hypertensive effect in a subject.

38. The composition of claim 30, further comprising a third amount of a compound selected from the group consisting of angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

39. A kit containing a first amount of an aldosterone receptor antagonist and a second amount of a renin inhibitor.

40. The kit of claim 39, comprising the first amount of the aldosterone receptor antagonist in a unit dosage form, and the second amount of a renin inhibitor in a unit dosage form.

41. The kit of claim 39, wherein the aldosterone receptor antagonist is an epoxy-steroidal-type compound characterized in having a 9α-,11α-substituted epoxy moiety.

42. The kit of claim 39, wherein the aldosterone receptor antagonist is selected from the group consisting of eplerenone and spironolactone.

43. The kit of claim 39, wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl) ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone(6α,7α,11α)-;
3′H-cyclopropa(6,7)pregna-4,6-diene-21-carboxylic acid,
9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

44. The kit of claim 39 wherein the renin inhibitor is selected from the group consisting of aliskiren, ditekiren, enalkiren, remikiren, terlakiren, and zankiren.

45. The kit of claim 44, wherein the aldosterone receptor antagonist is eplerenone.

46. A kit containing a first amount of an aldosterone receptor antagonist and a second amount of a renin inhibitor, wherein the first amount of the aldosterone receptor antagonist exhibits a release profile, determined using a suitable release profile test, in which more than about 20% by weight of the aldosterone receptor antagonist is released after initiation of the test.

47. The kit of claim 46, comprising the first amount of the aldosterone receptor antagonist in a unit dosage form, and the second amount of a renin inhibitor in a unit dosage form.

48. The kit of claim 46, wherein the aldosterone receptor antagonist is an epoxy-steroidal-type compound characterized in having a 9α-,11α-substituted epoxy moiety.

49. The kit of claim 46, wherein the aldosterone receptor antagonist is selected from the group consisting of eplerenone and spironolactone.

50. The kit of claim 46 wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl) ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone (6α,7α,11α)-;
3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

51. The kit of claim 46 wherein the renin inhibitor is selected from the group consisting of aliskiren, ditekiren, enalkiren, remikiren, terlakiren, and zankiren.

52. The kit of claim 51 wherein the aldosterone receptor antagonist is eplerenone.

53. The kit of claim 46 wherein at least about 30% by weight of the aldosterone receptor antagonist is released from the composition at about four hours after initiation of the test.
